SEQUENCE LISTING (37 sequences)

SEQ ID NO: 1 - DNA, 1725 bp, Respiratory syncytial virus
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaaggaaaataagtgtaatggaacagatgctaaggtaaaattgataaaacaagaattagataaatataaaaatgctgtaacagaattgcagttgctcatgcaaagcacaccaccaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaaataaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaagggatggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 2 - protein, 574 aa, Respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 3 - DNA, 1725 bp, Artificial Sequence
  F Protein 576
atggagctgctcatcttgaaggctaacgccattaccactatccttacagcggtgacgttctgctttgcatccggtcagaatattaccgaagagttctaccaatctacttgtagcgctgtctcaaaaggctatctgtcggccctccgtacaggatggtacacgagtgttatcaccatcgaattgtccaacattaaggagaacaagtgcaacggtactgacgcgaaggtaaagcttatcaaacaggaactggataagtacaagaacgcagtgacagagctccaattgctgatgcagtctacccccgctacgaataaccgcgctaggagagaacttccacgattcatgaactatactctcaataacgccaaaaagaccaacgtcacattgagcaaaaagcgtaagcgcaggtttctgggcttcctcctgggagttggttcagctattgcgtcgggcgtagccgtgagtaaagtccttcacttggagggagaagttaataagatcaagtccgcactcctgtctactaacaaagctgtggtcagcttgtcaaacggtgtatccgtgctgacctcgaaggttcttgacctcaaaaattacatcgataagcaattgctgccgattgtcaacaagcagagttgttctatcagcaatattgagacggtgatcgagttccaacagaaaaacaacagactcctggaaatcacacgtgagttttcagtaaatgccggcgttactacccccgtctccacgtacatgcttacaaactcggaattgctcagtctgattaacgacatgcctatcactaatgatcagaagaagcttatgtctaacaacgtgcaaattgtccgccagcaaagctattccatcatgtcaatcattaaagaggaagtgttggcgtacgtagttcagctcccactgtacggagtcatcgacaccccgtgctggaagcttcatacctcgcccttgtgtacgacaaatactaaagagggttctaacatttgcctcaccaggacggatcgaggctggtattgcgataacgctggaagtgtgagcttcttccctcaagcagaaacatgtaaggtacagtccaatagagttttttgcgacactatgaactcactgacccttccatctgaggtcaatttgtgtaacgtcgatatcttcaacccgaagtacgactgcaaaattatgacgtccaagacagatgtgtcgagtagcgtaatcacttcactcggtgccatcgtttcttgctacggcaagaccaaatgtacggcttccaataagaaccgtggaattatcaaaacattctcgaacggttgcgactatgtcagcaataagggcgtggacactgtgagtgtaggaaacaccctgtactacgttaacaagcaagaaggtaaatcactgtatgtcaagggcgagcccattatcaatttttacgatcctcttgtgttcccatccgacgagttcgatgcgtctatcagccaggtaaacgaaaagattaaccagtccttggcatttatccgcaaatcggacgagctcctgcacaatgttaacgccggaaagagtacgacaaacattatgatcactaccatcattatcgtcattatcgtgatccttttgtcactcattgctgtaggtctgcttttgtactgtaaagcgaggtctacgcccgttacactcagcaaggatcaactgtccggcatcaataacattgccttctcgaattaa

SEQ ID NO: 4 - protein, 574 aa, Artificial Sequence
  F Protein 576
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 5 - DNA, 1725 bp, Artificial Sequence
  F Protein 541
atggagctgctcatcttgaaggctaacgccattaccactatccttacagcggtgacgttctgctttgcatccggtcagaatattaccgaagagttctaccaatctacttgtagcgctgtctcaaaaggctatctgtcggccctccgtacaggatggtacacgagtgttatcaccatcgaattgtccaacattaaggagaacaagtgcaacggtactgacgcgaaggtaaagcttatcaaacaggaactggataagtacaagaacgcagtgacagagctccaattgctgatgcagtctacccccgctacgaataaccgcgctaggagagaacttccacgattcatgaactatactctcaataacgccaaaaagaccaacgtcacattgagcaaaaagcagaagcaacagtttctgggcttcctcctgggagttggttcagctattgcgtcgggcgtagccgtgagtaaagtccttcacttggagggagaagttaataagatcaagtccgcactcctgtctactaacaaagctgtggtcagcttgtcaaacggtgtatccgtgctgacctcgaaggttcttgacctcaaaaattacatcgataagcaattgctgccgattgtcaacaagcagagttgttctatcagcaatattgagacggtgatcgagttccaacagaaaaacaacagactcctggaaatcacacgtgagttttcagtaaatgccggcgttactacccccgtctccacgtacatgcttacaaactcggaattgctcagtctgattaacgacatgcctatcactaatgatcagaagaagcttatgtctaacaacgtgcaaattgtccgccagcaaagctattccatcatgtcaatcattaaagaggaagtgttggcgtacgtagttcagctcccactgtacggagtcatcgacaccccgtgctggaagcttcatacctcgcccttgtgtacgacaaatactaaagagggttctaacatttgcctcaccaggacggatcgaggctggtattgcgataacgctggaagtgtgagcttcttccctcaagcagaaacatgtaaggtacagtccaatagagttttttgcgacactatgaactcactgacccttccatctgaggtcaatttgtgtaacgtcgatatcttcaacccgaagtacgactgcaaaattatgacgtccaagacagatgtgtcgagtagcgtaatcacttcactcggtgccatcgtttcttgctacggcaagaccaaatgtacggcttccaataagaaccgtggaattatcaaaacattctcgaacggttgcgactatgtcagcaataagggcgtggacactgtgagtgtaggaaacaccctgtactacgttaacaagcaagaaggtaaatcactgtatgtcaagggcgagcccattatcaatttttacgatcctcttgtgttcccatccgacgagttcgatgcgtctatcagccaggtaaacgaaaagattaaccagtccttggcatttatccgcaaatcggacgagctcctgcacaatgttaacgccggaaagagtacgacaaacattatgatcactaccatcattatcgtcattatcgtgatccttttgtcactcattgctgtaggtctgcttttgtactgtaaagcgaggtctacgcccgttacactcagcaaggatcaactgtccggcatcaataacattgccttctcgaattaa

SEQ ID NO: 6 - protein, 574 aa, Artificial Sequence
  F protein 541
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKQKQQFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 7 - DNA, 1695 bp, Artificial Sequence
  F Protein 683
atggagctgctcatcttgaaggctaacgccattaccactatccttacagcggtgacgttctgctttgcatccggtcagaatattaccgaagagttctaccaatctacttgtagcgctgtctcaaaaggctatctgtcggccctccgtacaggatggtacacgagtgttatcaccatcgaattgtccaacattaaggagaacaagtgcaacggtactgacgcgaaggtaaagcttatcaaacaggaactggataagtacaagaacgcagtgacagagctccaattgctgatgcagtctacccccgctacgaataaccgcgctaggagagaacttccacgattcatgaactatactctcaataacgccaaaaagaccaacgtcacattgagcaaaaagcagaagcaacaggctattgcgtcgggcgtagccgtgagtaaagtccttcacttggagggagaagttaataagatcaagtccgcactcctgtctactaacaaagctgtggtcagcttgtcaaacggtgtatccgtgctgacctcgaaggttcttgacctcaaaaattacatcgataagcaattgctgccgattgtcaacaagcagagttgttctatcagcaatattgagacggtgatcgagttccaacagaaaaacaacagactcctggaaatcacacgtgagttttcagtaaatgccggcgttactacccccgtctccacgtacatgcttacaaactcggaattgctcagtctgattaacgacatgcctatcactaatgatcagaagaagcttatgtctaacaacgtgcaaattgtccgccagcaaagctattccatcatgtcaatcattaaagaggaagtgttggcgtacgtagttcagctcccactgtacggagtcatcgacaccccgtgctggaagcttcatacctcgcccttgtgtacgacaaatactaaagagggttctaacatttgcctcaccaggacggatcgaggctggtattgcgataacgctggaagtgtgagcttcttccctcaagcagaaacatgtaaggtacagtccaatagagttttttgcgacactatgaactcactgacccttccatctgaggtcaatttgtgtaacgtcgatatcttcaacccgaagtacgactgcaaaattatgacgtccaagacagatgtgtcgagtagcgtaatcacttcactcggtgccatcgtttcttgctacggcaagaccaaatgtacggcttccaataagaaccgtggaattatcaaaacattctcgaacggttgcgactatgtcagcaataagggcgtggacactgtgagtgtaggaaacaccctgtactacgttaacaagcaagaaggtaaatcactgtatgtcaagggcgagcccattatcaatttttacgatcctcttgtgttcccatccgacgagttcgatgcgtctatcagccaggtaaacgaaaagattaaccagtccttggcatttatccgcaaatcggacgagctcctgcacaatgttaacgccggaaagagtacgacaaacattatgatcactaccatcattatcgtcattatcgtgatccttttgtcactcattgctgtaggtctgcttttgtactgtaaagcgaggtctacgcccgttacactcagcaaggatcaactgtccggcatcaataacattgccttctcgaattaa

SEQ ID NO: 8 - protein, 564 aa, Artificial Sequence
  F protein 683
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKQKQQAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 9 - DNA, 1695 bp, Artificial Sequence
  F protein 622
atggagctgctcatcttgaaggctaacgccattaccactatccttacagcggtgacgttctgctttgcatccggtcagaatattaccgaagagttctaccaatctacttgtagcgctgtctcaaaaggctatctgtcggccctccgtacaggatggtacacgagtgttatcaccatcgaattgtccaacattaaggagaacaagtgcaacggtactgacgcgaaggtaaagcttatcaaacaggaactggataagtacaagaacgcagtgacagagctccaattgctgatgcagtctacccccgctacgaataaccgcgctaggagagaacttccacgattcatgaactatactctcaataacgccaaaaagaccaacgtcacattgagcaaaaagcgtaagcgcagggctattgcgtcgggcgtagccgtgagtaaagtccttcacttggagggagaagttaataagatcaagtccgcactcctgtctactaacaaagctgtggtcagcttgtcaaacggtgtatccgtgctgacctcgaaggttcttgacctcaaaaatcacatcgataagcaattgctgccgattgtcaacaagcagagttgttctatcagcaatattgagacggtgatcgagttccaacagaaaaacaacagactcctggaaatcacacgtgagttttcagtaaatgccggcgttactacccccgtctccacgtacatgcttacaaactcggaattgctcagtctgattaacgacatgcctatcactaatgatcagaagaagcttatgtctaacaacgtgcaaattgtccgccagcaaagctattccatcatgtcaatcattaaagaggaagtgttggcgtacgtagttcagctcccactgtacggagtcatcgacaccccgtgctggaagcttcatacctcgcccttgtgtacgacaaatactaaagagggttctaacatttgcctcaccaggacggatcgaggctggtattgcgataacgctggaagtgtgagcttcttccctcaagcagaaacatgtaaggtacagtccaatagagttttttgcgacactatgaactcactgacccttccatctgaggtcaatttgtgtaacgtcgatatcttcaacccgaagtacgactgcaaaattatgacgtccaagacagatgtgtcgagtagcgtaatcacttcactcggtgccatcgtttcttgctacggcaagaccaaatgtacggcttccaataagaaccgtggaattatcaaaacattctcgaacggttgcgactatgtcagcaataagggcgtggacactgtgagtgtaggaaacaccctgtactacgttaacaagcaagaaggtaaatcactgtatgtcaagggcgagcccattatcaatttttacgatcctcttgtgttcccatccgacgagttcgatgcgtctatcagccaggtaaacgaaaagattaaccagtccttggcatttatccgcaaatcggacgagctcctgcacaatgttaacgccggaaagagtacgacaaacattatgatcactaccatcattatcgtcattatcgtgatccttttgtcactcattgctgtaggtctgcttttgtactgtaaagcgaggtctacgcccgttacactcagcaaggatcaactgtccggcatcaataacattgccttctcgaattaa

SEQ ID NO: 10 - protein, 564 aa, Artificial Sequence
  F protein 622
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRAIASGVAVSKVLHLEGEVNKIKSALLSTNKAVVSLSNGVSVLTSKVLDLKNHIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 11 - DNA, 771 bp, Bovine respiratory syncytial virus
atggagacatacgtgaacaaactccatgaaggatcaacttacacagctgctgttcagtacaatgtcatagaaaaagatgatgatcctgcatctctcacaatatgggttcctatgttccaatcatccatctctgctgatttgcttataaaagaactaatcaatgtgaacatattagttcgacaaatttctactctgaaaggtccttcattgaagattatgataaactcaagaagtgctgtactagcccaaatgcccagcaaatttaccataagtgcaaatgtatcattggatgaacgaagcaaattagcatatgacataactactccttgtgaaattaaggcttgtagtttaacatgtttaaaggtgaaaaatatgctcacaactgtgaaagatctcaccatgaaaacattcaatcctacccatgagatcattgcactgtgtgaatttgaaaatatcatgacatccaaaagagttgttataccaactttcttaaggtcaatcaatgtaaaagcaaaggatttggactcactagagaatatagctaccacagagttcaaaaatgccatcactaatgctaaaattataccttatgctgggttggtattagttatcactgtaactgacaataaaggggcattcaagtacattaaaccacaaagtcaatttatagtagatcttggtgcatatctagagaaagagagcatatattatgtaactacaaattggaaacacacggccactaaattctccattaagcctatagaggactga

SEQ ID NO: 12 - protein, 256 aa, Bovine respiratory syncytial virus
METYVNKLHEGSTYTAAVQYNVIEKDDDPASLTIWVPMFQSSISADLLIKELINVNILVRQISTLKGPSLKIMINSRSAVLAQMPSKFTISANVSLDERSKLAYDITTPCEIKACSLTCLKVKNMLTTVKDLTMKTFNPTHEIIALCEFENIMTSKRVVIPTFLRSINVKAKDLDSLENIATTEFKNAITNAKIIPYAGLVLVITVTDNKGAFKYIKPQSQFIVDLGAYLEKESIYYVTTNWKHTATKFSIKPIED

SEQ ID NO: 13 - DNA, 771 bp, Artificial Sequence
  Codon Optimized Bovine RSV M
atggagacttacgtgaacaagctgcacgagggttccacctacaccgctgctgtgcagtacaacgtgatcgagaaggacgacgaccccgcttccctgaccatctgggtgcccatgttccagtcctccatctccgctgacctgctgatcaaggagctgatcaacgtcaacatcctcgtgcgtcagatctccaccctgaagggtcccagcctgaagatcatgatcaactcccgttccgctgtgctggctcagatgccctccaagttcaccatctccgccaacgtgtccctggacgagcgttccaagctggcttacgacatcaccaccccctgcgagatcaaggcttgctccctgacctgcctgaaggtcaagaacatgctgaccaccgtgaaggacctgaccatgaagaccttcaaccccacccacgagatcatcgctctgtgcgagttcgagaacatcatgacctccaagcgtgtggtcatccccaccttcctccgctccatcaacgtgaaggctaaggacctggactccctcgagaacatcgctaccaccgagttcaagaacgctatcaccaacgctaagatcatcccttacgctggcctggtgctggtcatcaccgtgaccgacaacaagggcgctttcaagtacatcaagccccagtcccagttcatcgtggacctgggcgcttacctcgagaaggagtccatctactacgtcaccaccaactggaagcacaccgctaccaagttctccatcaagcccatcgaggactaa

SEQ ID NO: 14 - protein, 256 aa, Artificial Sequence; identical to SEQ ID NO: 12
  Codon Optimized Bovine RSV M

SEQ ID NO: 15 - DNA, 1176 bp, Respiratory syncytial virus
atggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttga

SEQ ID NO: 16 - protein, 391 aa, Respiratory syncytial virus
MALSKVKLNDTLNKDQLLSSSKYTIQRSTGDSIDTPNYDVQKHINKLCGMLLITEDANHKFTGLIGMLYAMSRLGREDTIKILRDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKHPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVEL

SEQ ID NO: 17 - DNA, 1176 bp, Artificial Sequence
  Codon Optimized RSV N
atggctctgtccaaggtcaagctgaacgacaccctgaacaaggaccagctgctgtcctcctccaagtacaccatccagcgttccaccggtgactccatcgacacccccaactacgacgtgcagaagcacatcaacaagctgtgcggcatgctgctgatcaccgaggacgctaaccacaagttcaccggtctgatcggcatgctgtacgctatgtcccgtctgggtcgtgaggacaccatcaagatcctgcgtgacgctggttaccacgtgaaggctaacggtgtcgacgtgaccacccaccgtcaggacatcaacggcaaggagatgaagttcgaggtcctgaccctggcttccctgaccaccgagatccagatcaacatcgagatcgagtcccgtaagtcctacaagaagatgctgaaggagatgggcgaggtcgcccccgagtaccgtcacgactcccccgactgcggcatgatcatcctgtgcatcgctgctctcgtcatcaccaagctggctgctggtgaccgttccggtctgaccgctgtgatccgtcgtgctaacaacgtgctgaagaacgagatgaagcgctacaagggtctgctgcccaaggacatcgctaacagcttctacgaggtgttcgagaagcacccccacttcatcgacgtgttcgtgcacttcggtatcgctcagtcctccacccgtggtggttcccgtgtggagggcatcttcgctggtctgttcatgaacgcttacggtgctggccaggtcatgctgcgttggggtgtgctggctaagtccgtgaagaacatcatgctgggtcacgcttccgtgcaggctgagatggagcaggtggtggaggtgtacgagtacgctcagaagctgggcggcgaggctggtttctaccacatcctgaacaaccccaaggcttccctgctgtccctgacccagttcccccacttctcctccgtggtgctgggtaacgctgctggtctgggtatcatgggcgagtaccgtggcaccccccgtaaccaggacctgtacgacgctgctaaggcttacgccgagcagctcaaggagaacggcgtcatcaactactccgtgctggacctgaccgctgaggagctggaggctatcaagcaccagctgaaccccaaggacaacgacgtggagctgtaa

SEQ ID NO: 18 - protein, 391 aa, Artificial Sequence; identical to SEQ ID NO: 16
  Codon Optimized RSV N

SEQ ID NO: 19 - DNA, 1725 bp, Artificial Sequence
  F protein 368
atggagctgctcatcttgaaggctaacgccattaccactatccttacagcggtgacgttctgctttgcatccggtcagaatattaccgaagagttctaccaatctacttgtagcgctgtctcaaaaggctatctatcggccttacgtacaggatggtacacgagtgttatcaccatcgaactgtccaacattaaggagaataaatgcaacggtactgacgcgaaggtaaagctcatcaaacaggaattggataagtacaagaacgcagtgacagagcttcaactgctaatgcagtctaccccccctacgaataaccgcgctaggagagaactcccacgattcatgaactatactttaaataacgccaaaaagaccaacgtcacattgagcaaaaagcgtaagcgcaggtttctgggcttccttctcggagttggttcagctattgcgtcgggcgtagccgtgagtaaagtcttgcacctggagggagaagttaataagatcaagtccgcactcctgtctactaacaaagctgtggtcagcctatcaaacggtgtatccgtgttaacctcgaaggttcttgacttgaaaaattacatcgataagcaactcctgccgattgtcaacaagcagagttgttctatcagcaatattgagacggtgatcgagttccaacagaaaaacaacagattgctggaaatcacacgtgagttttcagtaaatgccggcgttactacccccgtctccacgtacatgctaacaaactcggaattacttagtctcattaacgacatgcctatcactaatgatcagaagaagttgatgtctaacaacgtgcaaattgtccgccagcaaagctattccatcatgtcaatcattaaagaggaagtgctggcgtacgtagttcagctcccactgtacggagtcatcgacaccccgtgctggaagctacatacctcgcccttatgtacgacaaatactaaagagggttctaacatttgccttaccaggacggatcgaggctggtattgcgataacgctggaagtgtgagcttcttccctcaagcagaaacatgtaaggtacagtccaatagagttttttgcgacactatgaactcattgaccctcccatctgagatcaatctgtgtaacgtcgatatcttcaacccgaagtacgactgcaaaattatgacgtccaagacagatgtgtcgagtagcgtaatcacttcactaggtgccatcgtttcttgctacggcaagaccaaatgtacggcttccaataagaaccgtggaattatcaaaacattctcgaacggttgcgactatgtcagcaataagggcatggacactgtgagtgtaggaaacaccttatactacgttaacaagcaagaaggtaaatcactttatgtcaagggcgagcccattatcaatttttacgatcctttggtgttcccatccgacgagttcgatgcgtctatcagccaggtaaacgaaaagattaaccagtccctcgcatttatccgcaaatcggacgagctgctacacaatgttaacgccggaaagagtacgacaaacattatgatcactaccatcattatcgtcattatcgtgatccttttgtcactcattgctgtaggtctgttactatactgtaaagcgaggtctacgcccgttacacttagcaaggatcaattgtccggcatcaataacattgccttctcgaattaa

SEQ ID NO: 20 - protein, 574 aa, Artificial Sequence; identical to SEQ ID NO: 2
  F protein 368

SEQ ID NO: 21 - protein, 560 aa, Artificial Sequence
  F protein 623
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRANNELPRFMNYTLNNAKKTNVTLSRRAIASGVAVSKVLHLEGEVNKIKSALLSTNKAVVSLSNGVSVLTSKVLDLKNHIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 22 - protein, 252 aa, Avian influenza virus
MSLLTEVETYVLSIIPSGPLKAEIAQKLEDVFAGKNTDLEALMEWLKTRPILSPLTKGILGFVFTLTVPSERGLQRRRFVQNALNGNGDPNNMDRAVKLYKKLKREITFHGAKEVSLSYSTGALASCMGLIYNRMGTVTTEVAFGLVCATCEQIADSQHRSHRQMATITNPLIRHENRMVLASTTAKAMEQMAGSSEQAAEAMEVANQARQMVQAMRTIGTHPNSSAGLRDNLLENLQAYQKRMGVQMQRFK

SEQ ID NO: 23 - protein, 4 aa, Artificial Sequence
  Secondary Cleavage Site
RARR

SEQ ID NO: 24 - protein, 6 aa, Artificial Sequence
  Primary Cleavage Site
KKRKRR

SEQ ID NO: 25 - DNA, 897 bp, Artificial Sequence
  Codon Optimized RSV G
atgtccaagaacaaagaccagcgtaccgctaagactctggagcgcacatgggatacgctcaatcacttgcttttcatctctagctgcctgtacaaactcaacttgaagtcagtggcccaaattaccctttcgatcctggcgatgattatcagtacttccctcatcattgcagctatcatttttatcgcctctgcgaatcataaggtcacacccacgaccgcaatcattcaggacgctactagccaaatcaaaaacacaacccctacgtatttgactcagaacccacaactgggtatttcaccgtcgaatcccagtgaaatcacctcccagatcacaactattcttgcctctaccacgcctggcgttaagagcacactccaatcaactaccgtaaagacgaaaaacacaactaccacccagacgcagccatccaagccgacaactaaacaaaggcagaacaagcccccttcgaagccaaataacgatttccacttcgaggtgtttaacttcgtcccgtgtagtatctgctctaataaccccacctgttgggctatttgcaaaagaatccctaacaagaagccaggaaaaaagacgacaactaaacccaccaagaagcctacgttgaaaacaactaagaaggacccgaaaccacaaaccacgaagagcaaagaagttcccacaactaagcctaccgaggaaccgacgatcaatacaactaagaccaacattatcacgacactgctcacttcaaataccactggtaacccagagctgacctcccagatggaaaccttccattcgacgagttctgagggcaaccccagcccttcccaagtatcaacaacttcggaatacccatctcagcccagtagccctccgaataccccacgacaataa

SEQ ID NO: 26 - protein, 298 aa, Artificial Sequence
  Codon Optimized RSV G
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 27 - protein, 65 aa, Respiratory syncytial virus
MGNTSITIEFTSKFWPYFTLIHMILTLISLLIIITIMIAILNKLSEHKTFCNNTLELGQMHQINT

SEQ ID NO: 28 - protein, 6 aa, Artificial Sequence
  Furin Recognition Site Mutation
KKQKQQ

SEQ ID NO: 29 - protein, 6 aa, Artificial Sequence
  Furin Recognition Site Mutation
GRRQQR

SEQ ID NO: 30 - protein, 4 aa, Artificial Sequence
  Furin Recognition Site Mutation
RAQQ

SEQ ID NO: 31 - protein, 6 aa, Artificial Sequence
  Furin Recognition Site Mutation
KKQKRQ

SEQ ID NO: 32 - protein, 11 aa, Artificial Sequence
  Primary Cleavage Site
LSKKRKRRFLG

SEQ ID NO: 33 - protein, 9 aa, Artificial Sequence
  Secondary Cleavage Site
NNRARRELP

SEQ ID NO: 34 - protein, 8 aa, Artificial Sequence
  Primary Cleavage Site Mutation
LSKKQKQQ

SEQ ID NO: 35 - protein, 25 aa, Artificial Sequence
  Palivizumab Epitope
NSELLSLINDMPITNDQKKLMSNNV

SEQ ID NO: 36 - protein, 4 aa, Artificial Sequence
  Furin Recognition Site Mutation
RANN

SEQ ID NO: 37 - protein, 11 aa, Artificial Sequence
  RSV F protein containing a Furin recognition site mutation
LSKKQKQQFLG

(19) United States
(12) Patent Application Publication
(10) Pub. No.: US 2014/0227309 A1
(43) Pub. Date: Aug. 14, 2014

(54) COMBINATION VACCINE FOR RESPIRATORY SYNCYTIAL VIRUS AND INFLUENZA

(71) Applicant: NOVAVAX, INC., Gaithersburg, MD (US)

(72) Inventors: Gale E. SMITH, Gaithersburg, MD (US); Greg GLENN, Gaithersburg, MD (US); Lou FRIES, Gaithersburg, MD (US); James F. YOUNG, Gaithersburg, MD (US)

(73) Assignee: NOVAVAX, INC., Gaithersburg, MD (US)

(21) Appl. No.: 14/177,406

(22) Filed: Feb. 11, 2014

Related U.S. Application Data
(60) Provisional application No. 61/763,309, filed on Feb. 11, 2013.
(60) Provisional application No. 61/875,327, filed on Sep. 9, 2013.

(51) Int. Cl. A61K 39/155; A61K 39/145

(52) U.S. Cl. CPC A61K 39/155; A61K 39/145
USPC 424/186.1

(57) ABSTRACT

The present disclosure is directed to compositions and methods for raising immune responses against influenza and respiratory synctial virus by administering combination immunogenic composition against both viruses at the same time. The combination compositions contain an RSV component and one, two, three, four, or more influenza components. The combination compositions provide a greater immune response than that obtained by separately administering the RSV and influenza components.

### CROSS-REFERENCE TO RELATED APPLICATIONS

This application claims priority to U.S. Provisional Application Nos. 61/763,309, filed Feb. 11, 2013, and 61/875,327, filed Sep. 9, 2013, each of which is incorporated in its entirety for all purposes.

This application incorporates the disclosures of the following applications in their entirety for all purposes: Ser. No. 13/269,107, filed Sep. 27, 2012, 61/015,440 filed Dec. 20, 2007, Ser. No. 11/582,540, filed Oct. 18, 2006 (U.S Patent Application Publication No. 2007/0184526), 60/727,513, filed Oct. 18, 2005; 60/780,847, filed Mar. 10, 2006; 60/800,006, filed May 15, 2006; 60/831,196, filed Jul. 17, 2006; 60/832,116, filed Jul. 21, 2006, 60/845,495, filed Sep. 19, 2006, Ser. No. 10/617,569, filed Jul. 11, 2003 (U.S Patent Application Publication No. 2005/0009008), Ser. No. 12/340,186 filed Dec. 19, 2008 (U.S Patent Application Publication No. 2010/0129401) and Ser. No. 12/689,826, filed Jan. 19, 2010 (U.S Patent Application Publication No. 2010/0184192).

### CROSS REFERENCE TO SEQUENCE LISTING

The contents of the text file submitted electronically herewith are incorporated herein by reference in their entirety: A computer readable format copy of the Sequence Listing (filename: NOVV_{—}53 Seq_List.txt, date recorded: Feb. 11, 2014; file size: 74 kilobytes).

### TECHNICAL FIELD

The present disclosure is generally related to immunogenic compositions, such as vaccines, for the treatment and/or prevention of infection by RSV and by influenza virus.

### BACKGROUND OF THE INVENTION

Respiratory syncytial virus (RSV) is a member of the genus Pneumovirus of the family Paramyxoviridae. Human RSV (HRSV) is the leading cause of severe lower respiratory tract disease in young children and is responsible for considerable morbidity and mortality in humans. RSV is also recognized as an important agent of disease in immunocompromised adults and in the elderly. Due to incomplete resistance to RSV in the infected host after a natural infection, RSV may infect multiple times during childhood and adult life.

This virus has a genome comprised of a single strand negative-sense RNA, which is tightly associated with viral protein to form the nucleocapsid. The viral envelope is composed of a plasma membrane derived lipid bilayer that contains virally encoded structural proteins. A viral polymerase is packaged with the virion and transcribes genomic RNA into mRNA. The RSV genome encodes three transmembrane structural proteins, F, G, and SH, two matrix proteins, M and M2, three nucleocapsid proteins N, P, and L, and two nonstructural proteins, NS1 and NS2.

Fusion of HRSV and cell membranes is thought to occur at the cell surface and is a necessary step for the transfer of viral ribonucleoprotein into the cell cytoplasm during the early stages of infection. This process is mediated by the fusion (F) protein, which also promotes fusion of the membrane of infected cells with that of adjacent cells to form a characteristic syncytia, which is both a prominent cytopathic effect and an additional mechanism of viral spread. Accordingly, neutralization of fusion activity is important in host immunity. Indeed, monoclonal antibodies developed against the F protein have been shown to neutralize virus infectivity and inhibit membrane fusion (Calder et al., 2000, Virology 271: 122-131).

The F protein of RSV shares structural features and limited, but significant amino acid sequence identity with F glycoproteins of other paramyxoviruses. It is synthesized as an inactive precursor of 574 amino acids (F0) that is cotranslationally glycosylated on asparagines in the endoplasmic reticulum, where it assembles into homo-oligomers. Before reaching the cell surface, the F0 precursor is cleaved by a protease into F2 from the N terminus and F1 from the C terminus. The F2 and F1 chains remain covalently linked by one or more disulfide bonds.

Immunoaffinity purified full-length F proteins have been found to accumulate in the form of micelles (also characterized as rosettes), similar to those observed with other full-length virus membrane glycoproteins (Wrigley et al., 1986, in Electron Microscopy of Proteins, Vol 5, p. 103-163, Academic Press, London). Under electron microscopy, the molecules in the rosettes appear either as inverted cone-shaped rods (˜70%) or lollipop-shaped (˜30%) structures with their wider ends projecting away from the centers of the rosettes. The rod conformational state is associated with an F glycoprotein in the pre-fusion inactivate state while the lollipop conformational state is associated with an F glycoprotein in the post-fusion, active state.

Electron micrography can be used to distinguish between the prefusion and postfusion (alternatively designated prefusogenic and fusogenic) conformations, as demonstrated by Calder et al., 2000, Virology 271:122-131. The prefusion conformation can also be distinguished from the fusogenic (postfusion) conformation by liposome association assays. Additionally, prefusion and fusogenic conformations can be distinguished using antibodies (e.g., monoclonal antibodies) that specifically recognize conformation epitopes present on one or the other of the prefusion or fusogenic form of the RSV F protein, but not on the other form. Such conformation epitopes can be due to preferential exposure of an antigenic determinant on the surface of the molecule. Alternatively, conformational epitopes can arise from the juxtaposition of amino acids that are non-contiguous in the linear polypeptide.

It has been shown previously that the F precursor is cleaved at two sites (site I, after residue 109 and site II, after residue 136), both preceded by motifs recognized by furin-like proteases. Site II is adjacent to a fusion peptide, and cleavage of the F protein at both sites is needed for membrane fusion (Gonzalez-Reyes et al., 2001, PNAS 98(17): 9859-9864). When cleavage is completed at both sites, it is believed that there is a transition from cone-shaped to lollipop-shaped rods.

### SUMMARY OF THE INVENTION

Provided herein are immunogenic compositions containing an RSV F component and at least one influenza component. Together, the RSV F and influenza components may be used to stimulate an immune response in an animal, such as a human, that protects against infection by RSV and influenza strains contained in the compositions.

The combination RSV F and Influenza VLP vaccines are well-tolerated and immunogenic in mice. Unexpectedly, the combination of components results in a heightened immune response against the viral antigens in the combination versus separately administering the components. Without being bound by mechanism, the immunogenicity data show that influenza antigens (possibly the HA portion) enhanced RSV F responses and conversely the RSV component increased HA responses, possibly due to the RSV buffer; for example, the lower pH than the influenza buffer, or the presence of histidine.

### BRIEF DESCRIPTION OF THE FIGURES

FIG. 1 illustrates preparing a disclosed combination composition by combining an RSV component and influenza component prior to administering the patient.

FIG. 2 describes specific antibodies induced against RSV by a disclosed combination composition compared to the RSV F and influenza components.

FIG. 3 describes neutralizing antibodies induced against RSV by a disclosed combination composition compared to the RSV F and influenza components.

FIG. 4 describes Palivizumab-competitive antibodies induced against RSV by a disclosed combination composition compared to the RSV F and influenza components.

FIG. 5 illustrates hemagglutination inhibition response induced by immunization against the A-California strain. The data compares the response induced by a combination RSV/trivalent influenza composition versus the RSV and trivalent influenza composition alone.

FIG. 6 illustrates hemagglutination inhibition response induced by immunization against the A/Victoria strain. The data compares the response induced by a combination RSV/trivalent influenza composition versus the RSV and trivalent influenza composition alone.

FIG. 7 illustrates hemagglutination inhibition response induced by immunization against the B/Wisconsin strain. The data compares the response induced by a combination RSV/trivalent influenza composition versus the RSV and trivalent influenza composition alone.

FIG. 8 illustrates anti-RSV F IgG response induced by immunization against the RSV F component. The data compares the response induced by sequential administration of RSV/TIV (trivalent influenza vaccine) composition components at different RSV antigen treatment levels in the presence or absence of aluminum phosphate adjuvant.

FIG. 9 illustrates the estimated level of palivizumab-like antibodies as measured by competitive ELISA. The data compares the response induced by a sequential administration of RSV and TIV influenza composition components at different RSV antigen treatment levels in the presence or absence of aluminum phosphate adjuvant and demonstrates that the induced immune response does not suffer from antigen interference.

FIG. 10 illustrates IgG binding to antigenic site II (Ag site II). The data compares the response induced by a sequential co administration of an RSV/TIV influenza composition at different RSV antigen treatment levels in the presence or absence of aluminum phosphate adjuvant and demonstrates that the compositions induce immune responses that do not suffer from antigen interference.

FIGS. 11A-B illustrates anti-RSV IgG responses in a mouse study. FIG. 11A illustrates the GMT of anti-RSV IgG response to an RSV-F composition, a quadrivalent influenza composition, and a combination vaccine containing both the RSV-F and quadrivalent compositions. FIG. 11B provides data in tabular form. Mice (n=10) were immunized on day 0 and 21 with quadrivalent influenza VLP (Q-Flu)+RSV F combination vaccine, RSV F or Q-Flu VLP vaccine alone. Sera were obtained from all the groups on day 35 to determine RSV F IgG response by ELISA as described in the method section 3.2. Data was analyzed using SoftMax pro software (Molecular Devices). A 4-parametric logistics (PL) curve was fitted to the data and titers were determined as the reciprocal value of the serum dilution that resulted in an OD450 of 1.0. The geometric mean titer (GMT) for each group are represented with the bar graph shown on figure. *p<0.05 compared with RSV F single vaccine

FIGS. 12A-B illustrates the Palivizumab-competitive antibody (PCA) response in a mouse study. FIG. 12A illustrates the PCA (μg/ml) of anti-RSV IgG response to an RSV-F composition, a quadrivalent influenza composition, and a combination vaccine containing both RSV-F and quadrivalent compositions. FIG. 12B provides data in tabular form. Mice (n=10) were immunized on day 0 and 21 with quadrivalent influenza VLP (Q-Flu)+RSV F combination vaccine, RSV F or Q-Flu VLP vaccine alone. Sera were obtained from all the groups on day 35 to determine palivizumab competitive antibody titers (PCA). PCA titers are reported as the reciprocal value of serum dilution that resulted in 50% inhibition of palivizumab monoclonal antibody binding to recombinant RSV F. Where 50% inhibition was not obtained, a titer of <20 was reported for the sample. The GMT of PCA in μg/ml are represented for each group with the bar graph shown on figure. *p<0.01 compared with RSV F single vaccine, +p<0.05 compared with RSV F single vaccine.

FIGS. 13A-B illustrates the RSV neutralizing antibody response in a mouse study. FIG. 13A illustrates the GMT of anti-RSV antibody response to an RSV-F composition, a quadrivalent influenza composition, and a combination vaccine containing both RSV-F and quadrivalent compositions. FIG. 13B provides data in tabular form. Mice (n=10) were immunized on day 0 and 21 with quadrivalent influenza VLP (Q-Flu)+RSV F combination vaccine, RSV F or Q-Flu VLP vaccine alone. Sera obtained from 35 following the immunization were used in microneutralization assay described in Examples 10 and 11. RSV neutralizing titers against RSV A2 providing 100% inhibition of (cytopathic effect) CPE were determined for each group with the bar graph shown on figure. The GMT are represented. *p<0.01 compared with RSV F single vaccine.

FIG. 14 shows the reagents used for performing hemagglutination inhibition (HAI) antibody assays.

FIGS. 15A-H illustrates anti-HA responses induced following administration of an RSV-F composition, a quadrivalent influenza composition, and a combination vaccine containing both RSV-F and quadrivalent compositions. The quadrivalent compositions contains VLPs with HA and NA proteins from four strains: A/California/04/09, A/Victoria/361/11, B/Brisbane/60/08 and B/Massachusetts/2/12. FIG. 15A shows the HAI analysis of the response to the A/California/04/09 (H1N1) influenza strain HA protein. FIG. 15B provides the A/California/04/09 influenza strain HAI data in tabular form. FIG. 15C shows the HAI analysis of the response to the A/Victoria/361/11 (H3N2) influenza strain HA protein. FIG. 15D provides A/Victoria/361/11 influenza strain HAI data in tabular form. FIG. 15E shows the HAI analysis of the response to the B/Brisbane/60/08 influenza strain HA protein. FIG. 15F provides B/Brisbane/60/08 influenza strain HAI data in tabular form. FIG. 15G shows the HAI analysis of the response to the B/Brisbane/60/08 influenza strain HA protein. FIG. 15H provides B/Brisbane/60/08 influenza strain HAI data in tabular form. Mice (n=10) were immunized on day 0 and 21 with quadrivalent influenza VLP (Q-Flu)+RSV F combination vaccine, RSV F or Q-Flu VLP vaccine alone. Day 35 sera were used to determine HAI titers to A/California, A/Victoria, B/Brisbane and B/Massachusetts. The geometric mean (GMT) for each group is represented.

FIGS. 16-19 show summary HAI titer data from Day 35 of a mouse study for strains: A/California (FIG. 16), A/Victoria (FIG. 17) B/Brisbane/60/08 (FIG. 18), and B/Massachusetts/2/12 (FIG. 19).

FIG. 20 shows data for Day 21 RSV IgG titers in a mouse study.

FIG. 21 shows data for Day 35 RSV IgG titers in a mouse study.

FIG. 22 shows data for Day 35 Competitive Palivizumab Antibody Titers in a mouse study.

FIG. 23 shows data for Day 35 Microneutralization Titers in a mouse study.

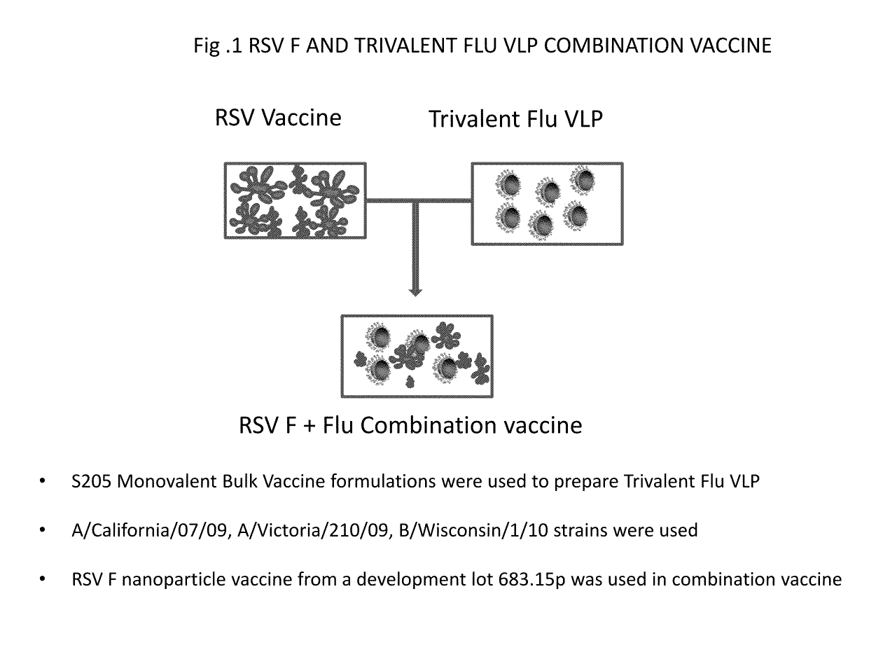

### DETAILED DESCRIPTION OF THE INVENTION

### Definitions

As used herein, the term “adjuvant” refers to a compound that, when used in combination with a specific immunogen in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

As used herein, the term “antigenic formulation” or “antigenic composition” refers to a preparation which, when administered to a vertebrate, especially a bird or a mammal, will induce an immune response.

As used herein, “about” means plus or minus 10% of the indicated value.

As used herein, the term “avian influenza virus” refers to influenza viruses found chiefly in birds but that can also infect humans or other animals. In some instances, avian influenza viruses may be transmitted or spread from one human to another. An avian influenza virus that infects humans has the potential to cause an influenza pandemic, i.e., morbidity and/or mortality in humans. A pandemic occurs when a new strain of influenza virus (a virus against which humans have no natural immunity) emerges, spreading beyond individual localities, possibly around the globe, and infecting many humans at once.

As used herein, an “effective dose” generally refers to that amount of a composition disclosed herein sufficient to induce immunity, to prevent and/or ameliorate an infection or to reduce at least one symptom of an infection or disease. An effective dose may also be the amount sufficient to enhance a subject's (e.g., a human's) own immune response against a subsequent exposure to an infectious agent or disease. Levels of immunity can be monitored, e.g., by measuring amounts of neutralizing secretory and/or serum antibodies, e.g., by plaque neutralization, complement fixation, enzyme-linked immunosorbent, or microneutralization assay, or by measuring cellular responses, such as, but not limited to cytotoxic T cells, antigen presenting cells, helper T cells, dendritic cells and/or other cellular responses. T cell responses can be monitored, e.g., by measuring, for example, the amount of CD4⁺ and CD8⁺ cells present using specific markers by fluorescent flow cytometry or T cell assays, such as but not limited to T-cell proliferation assay, T-cell cytotoxic assay, TETRAMER assay, and/or ELISPOT assay. In the case of a vaccine, an “effective dose” is one that prevents disease and/or reduces the severity of symptoms.

As used herein, the term “effective amount” refers to an amount of a composition disclosed herein necessary or sufficient to realize a desired biologic effect. An effective amount of the composition would be the amount that achieves a selected result, and such an amount could be determined as a matter of routine experimentation by a person skilled in the art. For example, an effective amount for preventing, treating and/or ameliorating an infection could be that amount necessary to cause activation of the immune system, resulting in the development of an antigen specific immune response. The term is also synonymous with “sufficient amount.”

As used herein, the term “expression” refers to the process by which polynucleic acids are transcribed into mRNA and translated into peptides, polypeptides, or proteins. If the polynucleic acid is derived from genomic DNA, expression may, if an appropriate eukaryotic host cell or organism is selected, include splicing of the mRNA. In the context of the present disclosure, the term also encompasses the yield of RSV F gene mRNA and RSV F proteins achieved following expression thereof.

As used herein, the term “F protein” or “Fusion protein” or “F protein polypeptide” or “Fusion protein polypeptide” refers to a polypeptide or protein having all or part of an amino acid sequence of an RSV Fusion protein polypeptide. Similarly, the term “G protein” or “G protein polypeptide” refers to a polypeptide or protein having all or part of an amino acid sequence of an RSV Attachment protein polypeptide. Numerous RSV Fusion and Attachment proteins have been described and are known to those of skill in the art. WO/2008/114149, which is herein incorporated by reference in its entirety, sets out exemplary F and G protein variants (for example, naturally occurring variants).

As used herein, the terms “immunogens” or “antigens” refer to substances such as proteins, peptides, and nucleic acids that are capable of eliciting an immune response. Both terms also encompass epitopes, and are used interchangeably.

As used herein the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons (IFN-γ), interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as VLPs of the disclosure, or can be administered separately. Either the protein or an expression vector encoding the protein can be administered to produce an immunostimulatory effect.

As used herein, the term “immunogenic formulation” refers to a preparation which, when administered to a vertebrate, e.g. a mammal, will induce an immune response.

As used herein, the term “infectious agent” refers to microorganisms that cause an infection in a vertebrate. Usually, the organisms are viruses, bacteria, parasites, protozoa and/or fungi.

As used herein, the terms “mutated,” “modified,” “mutation,” or “modification” indicate any modification of a nucleic acid and/or polypeptide which results in an altered nucleic acid or polypeptide. Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, an insertion, or a deletion of part or all of a gene. In some embodiments, the mutations are naturally-occurring. In other embodiments, the mutations are the results of artificial mutation pressure. In still other embodiments, the mutations in the RSV F proteins are the result of genetic engineering.

As used herein, the term “multivalent” refers to compositions which have one or more antigenic proteins/peptides or immunogens against multiple types or strains of infectious agents or diseases.

As used herein, the term “pharmaceutically acceptable vaccine” refers to a formulation that is in a form that is capable of being administered to a vertebrate and which induces a protective immune response sufficient to induce immunity to prevent and/or ameliorate an infection or disease, and/or to reduce at least one symptom of an infection or disease. Typically, the vaccine comprises a conventional saline or buffered aqueous solution medium in which the composition of the present disclosure is suspended or dissolved. In this form, the composition of the present disclosure can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

As used herein, the phrase “protective immune response” or “protective response” refers to an immune response mediated by antibodies against an infectious agent or disease, which is exhibited by a vertebrate (e.g., a human), that prevents or ameliorates an infection or reduces at least one disease symptom thereof. The compositions disclosed herein can stimulate the production of antibodies that, for example, neutralize infectious agents, blocks infectious agents from entering cells, blocks replication of the infectious agents, and/or protect host cells from infection and destruction.

As used herein, the term “vertebrate” or “subject” or “patient” refers to any member of the subphylum cordata, including, without limitation, humans and other primates, including non-human primates such as chimpanzees and other apes and monkey species. Farm animals such as cattle, sheep, pigs, goats and horses; domestic mammals such as dogs and cats; laboratory animals including rodents such as mice, rats (including cotton rats) and guinea pigs; birds, including domestic, wild and game birds such as chickens, turkeys and other gallinaceous birds, ducks, geese, and the like are also non-limiting examples. The terms “mammals” and “animals” are included in this definition. Both adult and newborn individuals are intended to be covered. In particular, infants and young children are appropriate subjects or patients for immunization against influenza and RSV.

As used herein, the term “virus-like particle” (VLP) refers to a structure that in at least one attribute resembles a virus but which has not been demonstrated to be infectious. Virus-like particles in accordance with the disclosure do not carry genetic information encoding for the proteins of the virus-like particles. In general, virus-like particles lack a viral genome and, therefore, are noninfectious. In addition, virus-like particles can often be produced in large quantities by heterologous expression and can be easily purified.

As used herein, the term “chimeric VLP” refers to VLPs that contain proteins, or portions thereof, from at least two different infectious agents (heterologous proteins). Usually, one of the proteins is derived from a virus that can drive the formation of VLPs from host cells. Examples, for illustrative purposes, are the BRSV M protein and/or the HRSV G or F proteins.

As used herein, the term “vaccine” refers to a composition containing one or more viral antigens used to induce a protective immune response against the virus.

### Compositions

The combination compositions described herein provide robust immune responses against RSV and multiple influenza viruses. Providing responses against multiple pathogens is advantageous in numerous respects. For example, it reduces costs associated with administration vaccines as part of an immunization program and patient compliance is improved because multiple immunizations are achieved via a single injection.

The compositions described herein contain an RSV component and one or more influenza components. The RSV component induces an immune response against RSV. The influenza components induce responses against influenza strains.

### RSV F Component

In an aspect of the disclosure, the RSV F component contains an RSV F protein. Suitable RSV F proteins and methods for their production are described in U.S. patent application Ser. No. 13/269,107.

The RSV F protein may comprise a modified or mutated amino acid sequence as compared to the wild-type RSV F protein (e.g. as exemplified in SEQ ID NO: 2; GenBank Accession No AAB59858).). In one embodiment, the RSV F protein contains a modification or mutation at the amino acid corresponding to position P102 of the wild-type RSV F protein (SEQ ID NO: 2). In another embodiment, the RSV F protein contains a modification or mutation at the amino acid corresponding to position 1379 of the wild-type RSV F protein (SEQ ID NO: 2). In another embodiment, the RSV F protein contains a modification or mutation at the amino acid corresponding to position M447 of the wild-type RSV F protein (SEQ ID NO: 2). In one embodiment, the RSV F protein contains two or more modifications or mutations at the amino acids corresponding to the positions described above. In another embodiment, the RSV F protein contains three modifications or mutations at the amino acids corresponding to the positions described above.

In one embodiment, the proline at position 102 is replaced with alanine. In another embodiment, the isoleucine at position 379 is replaced with valine. In yet another embodiment, the methionine at position 447 is replaced with valine. In certain embodiments, the RSV F protein contains two or more modifications or mutations at the amino acids corresponding to the positions described in these specific embodiments. In certain other embodiments, the RSV F protein contains three modifications or mutations at the amino acids corresponding to the positions described in these specific embodiments. In an exemplary embodiment, the RSV protein has the amino acid sequence described in SEQ ID NO: 4.

In one embodiment, the coding sequence of the RSV F protein is further optimized to enhance its expression in a suitable host cell. In one embodiment, the host cell is an insect cell. In an exemplary embodiment, the insect cell is an Sf9 cell.

In one embodiment, the coding sequence of the codon optimized RSV F gene is SEQ ID NO: 3. In another embodiment, the codon optimized RSV F protein has the amino acid sequence described in SEQ ID NO: 4.

In one embodiment, the RSV F protein further comprises at least one modification in the cryptic poly(A) site of F2. In another embodiment, the RSV F protein further comprises one or more amino acid mutations at the primary cleavage site (CS). In one embodiment, the RSV F protein contains a modification or mutation at the amino acid corresponding to position R133 of the wild-type RSV F protein (SEQ ID NO: 2) or the codon optimized RSV F protein (SEQ ID NO: 4). In another embodiment, the RSV F protein contains a modification or mutation at the amino acid corresponding to position R135 of the wild-type RSV F protein (SEQ ID NO: 2) or the codon optimized RSV F protein (SEQ ID NO: 4). In yet another embodiment, the RSV F protein contains a modification or mutation at the amino acid corresponding to position R136 of the wild-type RSV F protein (SEQ ID NO: 2) or the codon optimized RSV F protein (SEQ ID NO: 4).

In one embodiment, the arginine at position 133 is replaced with glutamine. In another embodiment, the arginine at position 135 is replaced with glutamine. In yet another embodiment, the arginine at position 136 is replaced with glutamine. In certain embodiments, the RSV F protein contains two, three, or more modifications or mutations at the amino acids corresponding to the positions described in these specific embodiments. In an exemplary embodiment, the RSV protein has the amino acid sequence described in SEQ ID NO: 6.

In another embodiment, the RSV F protein further comprises a deletion in the N-terminal half of the fusion domain corresponding to amino acids 137-146 of SEQ ID NO: 2, SEQ ID NO: 4, and SEQ ID NO: 6. In an exemplary embodiment, the RSV F protein has the amino acid sequence described in SEQ ID NO: 8. In an alternative embodiment, the RSV F protein has the amino acid sequence described in SEQ ID NO: 10.

The RSV F protein may be from various human strains, including strain A and strain B and non-human strains, including but not limited to bovine or avian RSV strains.

The RSV F protein may be in a virus-like particle (VLP). In some embodiments, the VLP further comprises one or more additional proteins. The RSV F component may contain additional proteins. For example, in one embodiment, the VLP further comprises a matrix (M) protein. In one embodiment, the M protein is derived from a human strain of RSV. In another embodiment, the M protein is derived from a bovine strain of RSV. In other embodiments, the matrix protein may be an M1 protein from an influenza virus strain. In one embodiment, the influenza virus strain is an avian influenza virus strain. In other embodiments, the M protein may be derived from a Newcastle Disease Virus (NDV) strain.

In additional embodiments, the VLP further comprises the RSV glycoprotein G. In another embodiment, the VLP further comprises the RSV glycoprotein SH. In yet another embodiment, the VLP further comprises the RSV nucleocapsid N protein.

The modified or mutated RSV F proteins may be used for the prevention and/or treatment of RSV infection. Thus, in another aspect, a method for eliciting an immune response against RSV is disclosed. The method involves administering an immunologically effective amount of a composition containing a modified or mutated RSV F protein to a subject, such as a human or animal subject.

Isolated nucleic acid sequences encoding RSV-F proteins are also provided. In an exemplary embodiment, the isolated nucleic acid encoding a modified or mutated RSV F protein is selected from the group consisting of SEQ ID NO: 3, SEQ ID NO: 5, SEQ ID NO: 7, or SEQ ID NO: 9. In addition to RSV F, other RSV proteins may be included. For example, the RSV component may further comprises one ore more additional RSV proteins, such as M, N, G, and SH.

### Influenza Component

As used herein, “influenza component” means a molecule containing at least one antigen capable of inducing a response against an influenza strain. In some aspects, the molecule is a protein or glycoprotein. In other aspects, the molecule is an influenza VLP. For example, in the trial discussed in Example 1, the administered vaccine combination contained three influenza components with each component being a VLP containing an HA and NA protein derived from strain A-Perth H3N2 S205, A-Cal H1N1, and B-Wisconsin. Each of the three VLPs contained an M1 protein derived from the same strain, A/Indonesia/5/05.

Compositions and methods for preparing and producing suitable influenza components are found in applications incorporated by reference above. The influenza VLP may contain one or more of an influenza matrix (M1) protein, an HA protein, and an NA protein. In some aspects, the influenza VLP contains all three proteins. Additional influenza proteins may also be included. An influenza M2 may be included in the VLP; however, preferably, at least one influenza VLP does not include an influenza M2 protein. More preferably, none of the influenza VLPs contain an M2 protein.

The influenza proteins may be obtained from any suitable influenza strain. In some aspects, the influenza proteins may all be from the same strain. In other aspects, each protein is from a different strain. In yet other aspects, the M1 protein is from one strain and the HA and NA proteins are from a second strain, which is a different strain than the M1 protein influenza strain. In still other aspects, the M1 protein and either the NA or HA protein, but not both, are from the same strain. In some aspects, the M1 protein is from an avian strain. In other aspects, the M1 protein is from a seasonal strain.

Exemplary strains include, but are not limited to, those having the following HA or NA proteins. The HA protein may be selected from the group consisting of H1, H2, H3, H4, H5, H6, H7, H8, H9, H10, H11, H12, H13, H14, H15, and H16. The NA protein may be selected from the group consisting of N1, N2, N3, N4, N5, N6, N7, N8 and N9. In a particular aspect, the HA and NA proteins are H5 and N1, respectively. In another aspect, the HA and NA proteins are H9 and N2, respectively. In yet another aspect, the HA and NA proteins are H7 and N9, respectively. The HA protein may exhibit hemagglutinin activity. The NA protein may exhibit neuraminidase activity.

In some aspects, a quadrivalent influenza composition may be used in the combination compositions. The quadrivalent influenza composition comprises four influenza VLP types, each containing an HA protein and an NA protein derived from a different influenza strain. In some aspects, the VLPs each contain an M1 protein derived from the A/Indonesia/5/05 influenza strain. In some aspects, the HA and NA proteins are derived from seasonal influenza strains identified by the FDA as useful in preventing influenza infection. In other aspects, a trivalent composition containing VLPs relevant for only three seasonal influenza strains may be used.

### Dosages

The disclosed compositions may be administered in to provide an effective dose. For example, the upper range of each influenza component delivered may be about: 1 μg, 2 μg, 3 μg, 4 μg, 5 μg, 6 μg, 7 μg, 8 μg, 9 μg, 10 μg, 15 μg, or 20 μg. The lower range of each influenza component delivered may be about: 0.5 μg 1 μg, 2 μg, 3 μg, 4 μg, 5 μg, 6 μg, 7 μg, 8 μg, 9 μg, 10 μg, or 15 μg. In some aspects the influenza component dose ranges from about 1 μg to about 3 μg. In other aspects, the influenza component dose ranges from about 3 μg to about 9 μg. The upper range of the RSV component delivered may be about: 1 μg, 2 μg, 3 μg, 4 μg, 5 μg, 6 μg, 7 μg, 8 μg, 9 μg, 10 μg, 15 μg, or 20 μg. The lower range of the RSV F component delivered may be about: 1 μg, 2 μg, 3 μg, 4 μg, 5 μg, 6 μg, 7 μg, 8 μg, 9 μg, 10 μg, 15 μg, or 20 μg. In some aspects the RSV component dose ranges from about 1 μg to about 3 μg. In other aspects, the RSV component dose ranges from about 3 μg to about 9 μg.

In other aspects, each influenza component may be present in a higher amount. For example, the upper range may be about: 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, 120 μg, 121 μg, 122 μg, 123 μg, 124 μg, 125 μg, 126 μg, 127 μg, 128 μg, 129 μg, 130 μg 131 μg, 132 μg, 133 μg, 134 μg, 135 μg, 136 μg, 137 μg, 138 μg, 139 μg, 140 μg, 141 μg, 142 μg, 143 μg, 144 μg, 145 μg, 146 μg, 147 μg, 148 μg, 149 μg, or 150 μg. The lower range for each influenza component may be about: 20 μg, 21 μg, 22 μg, 23 μg, 24 μg, 25 μg, 26 μg, 27 μg, 28 μg, 26 μg, 30 μg, 31 μg, 32 μg, 33 μg, 34 μg, 35 μg, 36 μg, 37 μg, 38 μg, 39 μg, 40 μg, 41 μg, 42, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, or 60 μg.

In other embodiments, such as in administration to humans, the upper range of the RSV component delivered may be about: 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, 120 μg, 121 μg, 122 μg, 123 μg, 124 μg, 125 μg, 126 μg, 127 μg, 128 μg, 129 μg, 130 μg 131 μg, 132 μg, 133 μg, 134 μg, 135 μg, 136 μg, 137 μg, 138 μg, 139 μg, 140 μg, 141 μg, 142 μg, 143 μg, 144 μg, 145 μg, 146 μg, 147 μg, 148 μg, 149 μg, or 150 μg. In some embodiments, such as in administration to humans, the lower range of the RSV F component delivered may be about: 20 μg, 21 μg, 22 μg, 23 μg, 24 μg, 25 μg, 26 μg, 27 μg, 28 μg, 26 μg, 30 μg, 31 μg, 32 μg, 33 μg, 34 μg, 35 μg, 36 μg, 37 μg, 38 μg, 39 μg, 40 μg, 41 μg, 42, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, or 60 μg. In some aspects, the RSV component dose ranges from about 40 μg to about 120 μg. In other aspects, the RSV component dose ranges from about 60 μg to about 90 μg.

### Adjuvants

As also well known in the art, the immunogenicity of a particular composition can be enhanced by the use of non-specific stimulators of the immune response, known as adjuvants. Adjuvants have been used experimentally to promote a generalized increase in immunity against unknown antigens (e.g., U.S. Pat. No. 4,877,611) Immunization protocols have used adjuvants to stimulate responses for many years, and as such, adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. The inclusion of any adjuvant described in Vogel et al., “A Compendium of Vaccine Adjuvants and Excipients (2^{nd} Edition),” herein incorporated by reference in its entirety for all purposes, is envisioned within the scope of this disclosure.

The compositions disclosed herein may be combined with a pharmaceutically acceptable adjuvant. Pharmaceutically acceptable adjuvants include but are not limited to aluminum based adjuvants, mineral salt adjuvants, tensoactive advjuvants, bacteria-derived adjuvants, emulsion adjuvants, liposome adjuvants, cytokine adjuvants, carbohydrate adjuvants, and DNA and RNA oligo adjuvants among others (see Petrovsky and Aguilar 2004, immunology and Cell biology 82, 488-496).

Exemplary adjuvants include complete Freund's adjuvant (a non-specific stimulator of the immune response containing killed Mycobacterium tuberculosis), and incomplete Freund's adjuvants. Other adjuvants comprise GMCSP, BCG, MDP compounds, such as thur-MDP and nor-MDP, CGP (MTP-PE), lipid A, and monophosphoryl lipid A (MPL), MF-59, RIBI, which contains three components extracted from bacteria, MPL, trehalose dimycolate (TDM), cell wall skeleton (CWS) in a 2% squalene/Tween® 80 emulsion, AS01, AS03 (squalene/tocopherol emulsion), AS04, AF3 (squalene o/w emulsion), glucopyranosyl lipid adjuvant-stable emulsion (GLA-SE), CoVaccine, Flagellin, and IC31 (dI:dC-TLR9 ago).

In some embodiments, the aluminum based adjuvants (Alum) may be aluminum phosphate or aluminum hydroxide. In certain aspects, 2% Alhydrogel (Al(OH)₃ is used. In some embodiments, the upper range of alum adjuvant that is administered is 200 μg, 300 μg, 400 μg, 500 μg, 600 μg, 700 μg, 800 μg, 900 μg, 1000 μg, 1100 μg, 1200 μg, 1300 μg, 1400 μg, 1500 μg, 1600 μg, 1700 μg, 1800 μg, 1900 μg, 2000 μg. In some embodiments, the lower range of alum adjuvant that is administered is 10 μg, 20 μg, 30 μg, 40 μg, 50 μg, 100 μg, 200 μg. In some embodiments, the amount of alum adjuvant administered ranges between about 200 μg to about 800 μg.

Preferred adjuvants include saponin-based adjuvants, particularly combinations of particular saponin fractions in ISCOM or Matrix format. In ISCOM format, the antigen is incorporated into the adjuvant cage-like structure. In Matrix format, the adjuvant is prepared first then combined with the antigenic compositions described herein to provide a desired formulation. Particularly suitable Matrix adjuvants include Matrix-M™ (AbISCO®-100, Isconova AB, Uppsala, Sweden), a mixture of Matrix-A™ and -C™ at the ratio of about 85:15. Briefly, Matrix-ATM and Matrix-C™ are prepared from separately purified fractions of Quillaja saponaria subsequently formulated with cholesterol and phospholipid into Matrix particles, then combined with antigen. See Reimer et al, “Matrix-M™ Adjuvant Induces Local Recruitment, Activation and Maturation of Central Immune Cells in Absence of Antigen,” PLoS ONE 7(7): e41451. doi:10.1371/journal.pone.0041451; See also U.S. Application Publication No. 2006/0121065. Other ratios of these fractions may also be used; for example, the ratio of Matrix-A to Matrix-C in the Matrix adjuvant composition may be about: 86:14, 87:13, 88:12, 89:11, 90:10, 91:9, 92:8, 93:7, 94:6, 95:5, or 96:4. Typically, the range is about 85-95 Matrix A to about 15-5 Matrix C. In some aspects, saponin fractions QS-7 and QS-21 may be used instead of Matrix A and Matrix C fractions. Exemplary QS-7 and QS-21 fractions and their use is described in U.S. Pat. Nos. 5,057,540; 6,231,859; 6,352,697; 6,524,584; 6,846,489; 7,776,343, and 8,173,141.

In one embodiment of the disclosure the adjuvant is a paucilamellar lipid vesicle having about two to ten bilayers arranged in the form of substantially spherical shells separated by aqueous layers surrounding a large amorphous central cavity free of lipid bilayers. Paucilamellar lipid vesicles may act to stimulate the immune response several ways, as non-specific stimulators, as carriers for the antigen, as carriers of additional adjuvants, and combinations thereof. Paucilamellar lipid vesicles act as non-specific immune stimulators when, for example, a vaccine is prepared by intermixing the antigen with the preformed vesicles such that the antigen remains extracellular to the vesicles. By encapsulating an antigen within the central cavity of the vesicle, the vesicle acts both as an immune stimulator and a carrier for the antigen. In another embodiment, the vesicles are primarily made of nonphospholipid vesicles. In other embodiments, the vesicles are Novasomes®. Novasomes® are paucilamellar nonphospholipid vesicles ranging from about 100 nm to about 500 nm. They comprise Brij 72, cholesterol, oleic acid and squalene. Novasomes have been shown to be an effective adjuvant for influenza antigens (see, U.S. Pat. Nos. 5,629,021, 6,387,373, and 4,911,928, herein incorporated by reference in their entireties for all purposes).

Immune Stimulators

The compositions of the disclosure can also be formulated with “immune stimulators.” These are the body's own chemical messengers (cytokines) to increase the immune system's response Immune stimulators include, but are not limited to, various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immunostimulatory molecules can be administered in the same formulation as the compositions of the disclosure, or can be administered separately. Either the protein or an expression vector encoding the protein can be administered to produce an immunostimulatory effect. Thus in one embodiment, the disclosure comprises antigentic and vaccine formulations comprising an adjuvant and/or an immune stimulator.

### Immune Responses

In addition to the compositions, the disclosure provides methods of inducing immune responses against RSV and multiple influenza strains. Compositions disclosed herein can induce substantial immunity in a vertebrate (e.g. a human) when administered to the vertebrate. Thus, in one embodiment, the disclosure provides a method of inducing substantial immunity to RSV virus infection and to influenza infection, or at least one symptom of each disease in a subject, comprising administering at least one effective dose of an RSV component and an influenza component. In another embodiment, the disclosure provides a method of vaccinating a mammal against RSV comprising administering to the mammal a protection-inducing amount of a modified or mutated RSV F protein, an RSV F micelle comprising a modified or mutated RSV F protein, or an RSV VLP comprising a modified or mutated RSV F protein, in combination with one or more influenza VLPs and/or one or more isolated influenza proteins.

In some aspects, the immune response comprises neutralizing antibodies. The titer of neutralizing antibodies may have an upper limit of about: 80, 90, 100, 125, 150, 175, 200, 225, or 250. The titer of neutralizing antibodies may have a lower limit of about: 70, 80, 90, 100, 125, 150, 175, 200, or 225. In some aspects, the range of titers is about 80 to about 250, about 100 to about 200, or about 150 to about 225. Neutralizing antibody titer may be measured by ELISA assay.

When multiple antigens are administered, the immune response to one or more can be reduced. This phenomenon is referred to as antigen interference. Preferably, the compositions disclosed herein induce an immune response that is not significantly different than that obtained when each antigen is administered separately. Thus, in preferred aspects, the compositions do not induce antigen interference. In other aspects, the combination composition immune response to each antigen is at least about 90%, 91%, 92%, 93%, 94%, 95%, 96%, 97%, 98% or 99% of the immune response obtained using each antigen alone.

Advantageously, combination compositions disclosed herein can enhance attributes of the immune response to RSV, compared to administration of the RSV component alone. For example, the immune response may comprise an anti-IgG response, a neutralizing anti-RSV response, and a palivizumab-competitive antibody response. In some aspects, the anti-IgG response is enhanced about 1.1-fold, 1.4-fold, about 1.6-fold, about 1.8-fold, about 2.0-fold, about 2.2-fold, about 2.4-fold, about 2.6-fold, about 2.8-fold, about 3.0-fold, about 3.2-fold, about 3.4-fold, about 3.6-fold, about 3.8-fold, about 4.0-fold, about 4.5-fold, or about 5.0-fold. In certain aspects, the fold increase in anti-RSV IgG response is about 2.0 to about 3.0. In other aspects, the fold increase in anti-RSV IgG response is about 1.4 to about 2.8. In some aspects, the enhanced response is measured 21 days after administration; in other aspects, the enhanced response is measured 35 days after administration.

The neutralizing anti-RSV response may also be enhanced compared to administration of the RSV component alone. In some aspects, the neutralizing anti-RSV response is enhanced about 1.1-fold, 1.4-fold, about 1.6-fold, about 1.8-fold, about 2.0-fold, about 2.2-fold, about 2.4-fold, about 2.6-fold, about 2.8-fold, about 3.0-fold, about 3.2-fold, about 3.4-fold, about 3.6-fold, about 3.8-fold, about 4.0-fold, about 4.5-fold, or about 5.0-fold. In certain aspects, the fold increase in neutralizing anti-RSV response is 1.1 to about 2.0. In other aspects, the fold increase in neutralizing anti-RSV response is about 2.3 to about 2.8. In some aspects, the enhanced response is measured 21 days after administration; in other aspects, the enhanced response is measured 35 days after administration.

The palivizumab-competitive antibody response may also be enhanced compared to administration of the RSV component alone. In some aspects, the palivizumab-competitive antibody response is enhanced about 2.0-fold, about 2.2-fold, about 2.4-fold, about 2.6-fold, about 2.8-fold, about 3.0-fold, about 3.2-fold, about 3.4-fold, about 3.6-fold, about 3.8-fold, about 4.0-fold, about 4.5-fold, or about 5.0-fold. In certain aspects, the fold increase in palivizumab-competitive antibody response is about 2.0 to about 3.0. In other aspects, the fold increase in palivizumab-competitive antibody response is about 2.3 to about 2.8. In some aspects, the enhanced response is measured 21 days after administration; in other aspects, the enhanced response is measured 35 days after administration.

### Identification and Cloning of Proteins and Variants

The disclosure also encompasses variants of the proteins expressed on or in the VLPs. The variants may contain alterations in the amino acid sequences of the constituent proteins. The term “variant” with respect to a protein refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software.

Natural variants can occur due to mutations in the proteins. These mutations may lead to antigenic variability within individual groups of infectious agents, for example influenza. Thus, a person infected with, for example, an influenza strain develops antibody against that virus, as newer virus strains appear, the antibodies against the older strains no longer recognize the newer virus and re-infection can occur. The disclosure encompasses all antigenic and genetic variability of proteins from infectious agents for making VLPs.

General texts which describe molecular biological techniques, which are applicable to the present disclosure, such as cloning, mutation, cell culture and the like, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the cloning and mutating F and/or G molecules of RSV, etc. Thus, the disclosure also encompasses using known methods of protein engineering and recombinant DNA technology to improve or alter the characteristics of the proteins expressed on or in the VLPs of the disclosure. Various types of mutagenesis can be used to produce and/or isolate variant nucleic acids that encode for protein molecules and/or to further modify/mutate the proteins in or on the VLPs of the disclosure. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, is also included in the present disclosure. In one embodiment, mutagenesis can be guided by known information of the naturally occurring molecule or altered or mutated naturally occurring molecule, e.g., sequence, sequence comparisons, physical properties, crystal structure or the like.

The disclosure further comprises protein variants which show substantial biological activity, e.g., able to elicit an effective antibody response when expressed on or in VLPs of the disclosure. Such variants include deletions, insertions, inversions, repeats, and substitutions selected according to general rules known in the art so as have little effect on activity.

Methods of cloning the proteins are known in the art. For example, the gene encoding a specific RSV protein can be isolated by RT-PCR from polyadenylated mRNA extracted from cells which had been infected with a RSV virus. The resulting product gene can be cloned as a DNA insert into a vector. The term “vector” refers to the means by which a nucleic acid can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In many, but not all, common embodiments, the vectors of the present disclosure are plasmids or bacmids.

Thus, the disclosure comprises nucleotides that encode proteins, including chimeric molecules, cloned into an expression vector that can be expressed in a cell that induces the formation of VLPs of the disclosure. An “expression vector” is a vector, such as a plasmid that is capable of promoting expression, as well as replication of a nucleic acid incorporated therein. Typically, the nucleic acid to be expressed is “operably linked” to a promoter and/or enhancer, and is subject to transcription regulatory control by the promoter and/or enhancer. In one embodiment, the nucleotides encode for a modified or mutated RSV F protein (as discussed above). In another embodiment, the vector further comprises nucleotides that encode the M and/or G RSV proteins. In another embodiment, the vector further comprises nucleotides that encode the M and/or N RSV proteins. In another embodiment, the vector further comprises nucleotides that encode the M, G and/or N RSV proteins. In another embodiment, the vector further comprises nucleotides that encode a BRSV M protein and/or N RSV proteins. In another embodiment, the vector further comprises nucleotides that encode a BRSV M and/or G protein, or influenza HA and/or NA protein. In another embodiment, the nucleotides encode a modified or mutated RSV F and/or RSV G protein with an influenza HA and/or NA protein. In another embodiment, the expression vector is a baculovirus vector.

In addition, the nucleotides can be sequenced to ensure that the correct coding regions were cloned and do not contain any unwanted mutations. The nucleotides can be subcloned into an expression vector (e.g. baculovirus) for expression in any cell. The above is only one example of how the RSV viral proteins can be cloned. A person with skill in the art understands that additional methods are available and are possible.

The disclosure also provides for constructs and/or vectors that comprise RSV nucleotides that encode for RSV structural genes, including F, M, G, N, SH, or portions thereof, and/or any chimeric molecule described above. The vector may be, for example, a phage, plasmid, viral, or retroviral vector. The constructs and/or vectors that comprise RSV structural genes, including F, M, G, N, SH, or portions thereof, and/or any chimeric molecule described above, should be operatively linked to an appropriate promoter, such as the AcMNPV polyhedrin promoter (or other baculovirus), phage lambda PL promoter, the E. coli lac, phoA and tac promoters, the SV40 early and late promoters, and promoters of retroviral LTRs are non-limiting examples. Other suitable promoters will be known to the skilled artisan depending on the host cell and/or the rate of expression desired. The expression constructs will further contain sites for transcription initiation, termination, and, in the transcribed region, a ribosome-binding site for translation. The coding portion of the transcripts expressed by the constructs will preferably include a translation initiating codon at the beginning and a termination codon appropriately positioned at the end of the polypeptide to be translated.

Expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Among vectors preferred are virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with the disclosure comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBac1 pWINEO, pSV2CAT, pOG44, pXT1 and pSG, pSVK3, pBPV, pMSG, and pSVL. Other suitable vectors will be readily apparent to the skilled artisan. In one embodiment, the vector that comprises nucleotides encoding for RSV genes, including modified or mutated RSV F genes, as well as genes for M, G, N, SH or portions thereof, and/or any chimeric molecule described above, is pFastBac.

The recombinant constructs mentioned above could be used to transfect, infect, or transform and can express RSV proteins, including a modified or mutated RSV F protein and at least one immunogen. In one embodiment, the recombinant construct comprises a modified or mutated RSV F, M, G, N, SH, or portions thereof, and/or any molecule described above, into eukaryotic cells and/or prokaryotic cells. Thus, the disclosure provides for host cells which comprise a vector (or vectors) that contain nucleic acids which code for RSV structural genes, including a modified or mutated RSV F; and at least one immunogen such as but not limited to RSV G, N, and SH, or portions thereof, and/or any molecule described above, and permit the expression of genes, including RSV F, G, N, M, or SH or portions thereof, and/or any molecule described above in the host cell under conditions which allow the formation of VLPs.

Among eukaryotic host cells are yeast, insect, avian, plant, C. elegans (or nematode) and mammalian host cells. Non limiting examples of insect cells are, Spodoptera frugiperda (Sf) cells, e.g. Sf9, Sf21, Trichoplusia ni cells, e.g. High Five cells, and Drosophila S2 cells. Examples of fungi (including yeast) host cells are S. cerevisiae, Kluyveromyces lactis (K. lactis), species of Candida including C. albicans and C. glabrata, Aspergillus nidulans, Schizosaccharomyces pombe (S. pombe), Pichia pastoris, and Yarrowia lipolytica. Examples of mammalian cells are COS cells, baby hamster kidney cells, mouse L cells, LNCaP cells, Chinese hamster ovary (CHO) cells, human embryonic kidney (HEK) cells, and African green monkey cells, CV1 cells, HeLa cells, MDCK cells, Vero and Hep-2 cells. Xenopus laevis oocytes, or other cells of amphibian origin, may also be used. Examples of prokaryotic host cells include bacterial cells, for example, E. coli, B. subtilis, Salmonella typhi and mycobacteria.

Vectors, e.g., vectors comprising polynucleotides of encoding proteins described herein can be transfected into host cells according to methods well known in the art. For example, introducing nucleic acids into eukaryotic cells can be by calcium phosphate co-precipitation, electroporation, microinjection, lipofection, and transfection employing polyamine transfection reagents. In one embodiment, the vector is a recombinant baculovirus. In another embodiment, the recombinant baculovirus is transfected into a eukaryotic cell. In a preferred embodiment, the cell is an insect cell. In another embodiment, the insect cell is a Sf9 cell.

This disclosure also provides for constructs and methods that will increase the efficiency of VLP production. For example, the addition of leader sequences to a protein can improve the efficiency of protein transporting within the cell. For example, a heterologous signal sequence such as those derived from an insect cell gene can be fused to a protein. In an embodiment, the signal peptide is the chitinase signal sequence, which works efficiently in baculovirus expression systems.

Another method to increase efficiency of VLP production is to codon optimize the nucleotides that encode RSV proteins. For examples of codon optimizing nucleic acids for expression in Sf9 cell see SEQ ID Nos: 3, 5, 7, 9, 13, 17, 19, and 25.

The disclosure also provides for methods of producing VLPs. In some aspects, the methods comprising expressing RSV genes including a modified or mutated RSV F protein, and at least one additional protein, including but not limited to RSV M, G, N, SH, or portions thereof, and/or any chimeric or heterologous molecules described above under conditions that allow VLP formation. In other aspects methods for producing influenza VLPs are provided. Additional disclosure regarding influenza VLPs are found in U.S Patent Application Publication Nos. 2005/0009008, 2010/0129401 and 2010/0184192, which are incorporated herein for all purposes. Depending on the expression system and host cell selected, the VLPs are produced by growing host cells transformed by an expression vector under conditions whereby the recombinant proteins are expressed and VLPs are formed. In one embodiment, the disclosure comprises a method of producing a VLP, comprising transfecting vectors encoding at least one modified or mutated RSV F protein into a suitable host cell and expressing the modified or mutated RSV F protein under conditions that allow VLP formation. In another embodiment, the eukaryotic cell is selected from the group consisting of, yeast, insect, amphibian, avian or mammalian cells. The selection of the appropriate growth conditions is within the skill or a person with skill of one of ordinary skill in the art.

Methods to grow cells engineered to produce VLPs of the disclosure include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, the bioreactor is a stainless steel chamber. In another embodiment, the bioreactor is a pre-sterilized plastic bag (e.g. Cellbag, Wave Biotech, Bridgewater, N.J.). In other embodiment, the pre-sterilized plastic bags are about 50 L to 1000 L bags.

The VLPs are then isolated using methods that preserve the integrity thereof, such as by gradient centrifugation, e.g., cesium chloride, sucrose and iodixanol, as well as standard purification techniques including, e.g., ion exchange and gel filtration chromatography.

The following is an example of how VLPs of the disclosure can be made, isolated and purified. Usually VLPs are produced from recombinant cell lines engineered to create VLPs when the cells are grown in cell culture (see above). A person of skill in the art would understand that there are additional methods that can be utilized to make and purify VLPs of the disclosure, thus the disclosure is not limited to the method described.

Production of VLPs of the disclosure can start by seeding Sf9 cells (non-infected) into shaker flasks, allowing the cells to expand and scaling up as the cells grow and multiply (for example from a 125-ml flask to a 50 L Wave bag). The medium used to grow the cell is formulated for the appropriate cell line (preferably serum free media, e.g. insect medium ExCell-420, JRH). Next, the cells are infected with recombinant baculovirus at the most efficient multiplicity of infection (e.g. from about 1 to about 3 plaque forming units per cell). Once infection has occurred, the modified or mutated RSV F protein, M, G, N, SH, or portions thereof, and/or any chimeric or heterologous molecule described above, are expressed from the virus genome, self assemble into VLPs and are secreted from the cells approximately 24 to 72 hours post infection. Usually, infection is most efficient when the cells are in mid-log phase of growth (4-8×10⁶ cells/ml) and are at least about 90% viable.

VLPs of the disclosure can be harvested approximately 48 to 96 hours post infection, when the levels of VLPs in the cell culture medium are near the maximum but before extensive cell lysis. The Sf9 cell density and viability at the time of harvest can be about 0.5×10⁶ cells/ml to about 1.5×10⁶ cells/ml with at least 20% viability, as shown by dye exclusion assay. Next, the medium is removed and clarified. NaCl can be added to the medium to a concentration of about 0.4 to about 1.0 M, preferably to about 0.5 M, to avoid VLP aggregation. The removal of cell and cellular debris from the cell culture medium containing VLPs of the disclosure can be accomplished by tangential flow filtration (TFF) with a single use, pre-sterilized hollow fiber 0.5 or 1.00 μm filter cartridge or a similar device.

Next, VLPs in the clarified culture medium can be concentrated by ultra-filtration using a disposable, pre-sterilized 500,000 molecular weight cut off hollow fiber cartridge. The concentrated VLPs can be diafiltrated against 10 volumes pH 7.0 to 8.0 phosphate-buffered saline (PBS) containing 0.5 M NaCl to remove residual medium components.

The concentrated, diafiltered VLPs can be furthered purified on a 20% to 60% discontinuous sucrose gradient in pH 7.2 PBS buffer with 0.5 M NaCl by centrifugation at 6,500×g for 18 hours at about 4° C. to about 10° C. Usually VLPs will form a distinctive visible band between about 30% to about 40% sucrose or at the interface (in a 20% and 60% step gradient) that can be collected from the gradient and stored. This product can be diluted to comprise 200 mM of NaCl in preparation for the next step in the purification process. This product contains VLPs and may contain intact baculovirus particles.

Further purification of VLPs can be achieved by anion exchange chromatography, or 44% isopycnic sucrose cushion centrifugation. In anion exchange chromatography, the sample from the sucrose gradient (see above) is loaded into column containing a medium with an anion (e.g. Matrix Fractogel EMD TMAE) and eluded via a salt gradient (from about 0.2 M to about 1.0 M of NaCl) that can separate the VLP from other contaminates (e.g. baculovirus and DNA/RNA). In the sucrose cushion method, the sample comprising the VLPs is added to a 44% sucrose cushion and centrifuged for about 18 hours at 30,000 g. VLPs form a band at the top of 44% sucrose, while baculovirus precipitates at the bottom and other contaminating proteins stay in the 0% sucrose layer at the top. The VLP peak or band is collected.

The intact baculovirus can be inactivated, if desired. Inactivation can be accomplished by chemical methods, for example, formalin or fl-propiolactone (BPL). Removal and/or inactivation of intact baculovirus can also be largely accomplished by using selective precipitation and chromatographic methods known in the art, as exemplified above. Methods of inactivation comprise incubating the sample containing the VLPs in 0.2% of BPL for 3 hours at about 25° C. to about 27° C. The baculovirus can also be inactivated by incubating the sample containing the VLPs at 0.05% BPL at 4° C. for 3 days, then at 37° C. for one hour.

After the inactivation/removal step, the product comprising VLPs can be run through another diafiltration step to remove any reagent from the inactivation step and/or any residual sucrose, and to place the VLPs into the desired buffer (e.g. PBS). The solution comprising VLPs can be sterilized by methods known in the art (e.g. sterile filtration) and stored in the refrigerator or freezer.

The above techniques can be practiced across a variety of scales. For example, T-flasks, shake-flasks, spinner bottles, up to industrial sized bioreactors. The bioreactors can comprise either a stainless steel tank or a pre-sterilized plastic bag (for example, the system sold by Wave Biotech, Bridgewater, N.J.). A person with skill in the art will know what is most desirable for their purposes.

Expansion and production of baculovirus expression vectors and infection of cells with recombinant baculovirus to produce recombinant VLPs can be accomplished in insect cells, for example Sf9 insect cells as previously described. In one embodiment, the cells are SF9 infected with recombinant baculovirus engineered to produce VLPs.

### Pharmaceutical or Vaccine Formulations and Administration

The pharmaceutical compositions useful herein contain a pharmaceutically acceptable carrier, including any suitable diluent or excipient. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful as a vaccine and/or antigenic compositions for inducing a protective immune response in a vertebrate. The compositions contain an RSV component and at least one influenza component.

The disclosure encompasses a pharmaceutically acceptable vaccine composition comprising VLPs comprising an RSV F protein, and at least one additional protein, including but not limited to RSV M, G, N, SH, or portions thereof, and/or any chimeric or heterologous molecules described above, in combination with at least one influenza antigen. In one embodiment, the pharmaceutically acceptable vaccine composition comprises VLPs comprising at least one RSV F protein and at least one additional immunogen. In another embodiment, the pharmaceutically acceptable vaccine composition comprises VLPs comprising at least one RSV F protein and at least one RSV M protein. In another embodiment, the pharmaceutically acceptable vaccine composition comprises VLPs comprising at least one RSV F protein and at least one BRSV M protein. In another embodiment, the pharmaceutically acceptable vaccine composition comprises VLPs comprising at least one RSV F protein and at least one influenza M1 protein. In another embodiment, the pharmaceutically acceptable vaccine composition comprises VLPs comprising at least one modified or mutated RSV F protein and at least one avian influenza VLP.

In another embodiment, the pharmaceutically acceptable vaccine composition comprises VLPs further comprising an RSV G protein, including but not limited to a HRSV, BRSV or avian RSV G protein. In another embodiment, the pharmaceutically acceptable vaccine composition comprises VLPs further comprising RSV N protein, including but not limited to a HRSV, BRSV or avian RSV N protein. In another embodiment, the pharmaceutically acceptable vaccine composition comprises VLPs further comprising RSV SH protein, including but not limited to a HRSV, BRSV or avian RSV SH protein.

In another embodiment, the disclosure encompasses a pharmaceutically acceptable vaccine composition comprising chimeric VLPs such as VLPs comprising BRSV M and a modified or mutated RSV F protein and/or G, H, or SH protein from a RSV and optionally HA or NA protein derived from an influenza virus, wherein the HA or NA protein is a fused to the transmembrane domain and cytoplasmic tail of RSV F and/or G protein.

The disclosure also encompasses a pharmaceutically acceptable vaccine composition comprising modified or mutated RSV F protein, an RSV F micelle comprising a modified or mutated RSV F protein, or a VLP comprising a modified or mutated RSV F protein as described above.

In one embodiment, the pharmaceutically acceptable vaccine composition comprises VLPs comprising a modified or mutated RSV F protein and at least one additional protein. In another embodiment, the pharmaceutically acceptable vaccine composition comprises VLPs further comprising RSV M protein, such as but not limited to a BRSV M protein. In another embodiment, the pharmaceutically acceptable vaccine composition comprises VLPs further comprising RSV G protein, including but not limited to a HRSV G protein. In another embodiment, the pharmaceutically acceptable vaccine composition comprises VLPs further comprising RSV N protein, including but not limited to a HRSV, BRSV or avian RSV N protein. In another embodiment, the pharmaceutically acceptable vaccine composition comprises VLPs further comprising RSV SH protein, including but not limited to a HRSV, BRSV or avian RSV SH protein. In another embodiment, the pharmaceutically acceptable vaccine composition comprises VLPs comprising BRSV M protein and F and/or G protein from HRSV group A. In another embodiment, the pharmaceutically acceptable vaccine composition comprises VLPs comprising BRSV M protein and F and/or G protein from HRSV group B. In another embodiment, the disclosure encompasses a pharmaceutically acceptable vaccine composition comprising chimeric VLPs such as VLPs comprising chimeric M protein from a BRSV and optionally HA protein derived from an influenza virus, wherein the M protein is fused to the influenza HA protein. In another embodiment, the disclosure encompasses a pharmaceutically acceptable vaccine composition comprising chimeric VLPs such as VLPs comprising BRSV M, and a chimeric F and/or G protein from a RSV and optionally HA protein derived from an influenza virus, wherein the chimeric influenza HA protein is fused to the transmembrane domain and cytoplasmic tail of RSV F and/or G protein. In another embodiment, the disclosure encompasses a pharmaceutically acceptable vaccine composition comprising chimeric VLPs such as VLPs comprising BRSV M and a chimeric F and/or G protein from a RSV and optionally HA or NA protein derived from an influenza virus, wherein the HA or NA protein is a fused to the transmembrane domain and cytoplasmic tail of RSV F and/or G protein.

The disclosure also encompasses a pharmaceutically acceptable vaccine composition comprising a chimeric VLP that comprises at least one RSV protein. In one embodiment, the pharmaceutically acceptable vaccine composition comprises VLPs comprising a modified or mutated RSV F protein and at least one immunogen from a heterologous infectious agent or diseased cell. In another embodiment, the immunogen from a heterologous infectious agent is a viral protein. In another embodiment, the viral protein from a heterologous infectious agent is an envelope associated protein. In another embodiment, the viral protein from a heterologous infectious agent is expressed on the surface of VLPs. In another embodiment, the protein from an infectious agent comprises an epitope that will generate a protective immune response in a vertebrate.

The disclosure also encompasses a kit for immunizing a vertebrate, such as a human subject, comprising VLPs that comprise at least one RSV protein. In one embodiment, the kit comprises VLPs comprising a modified or mutated RSV F protein. In one embodiment, the kit further comprises a RSV M protein such as a BRSV M protein. In another embodiment, the kit further comprises a RSV G protein. In another embodiment, the disclosure encompasses a kit comprising VLPs which comprises a chimeric M protein from a BRSV and optionally HA protein derived from an influenza virus, wherein the M protein is fused to the BRSV M. In another embodiment, the disclosure encompasses a kit comprising VLPs which comprises a chimeric M protein from a BRSV, a RSV F and/or G protein and an immunogen from a heterologous infectious agent. In another embodiment, the disclosure encompasses a kit comprising VLPs which comprises a M protein from a BRSV, a chimeric RSV F and/or G protein and optionally HA protein derived from an influenza virus, wherein the HA protein is fused to the transmembrane domain and cytoplasmic tail of RSV F or G protein. In another embodiment, the disclosure encompasses a kit comprising VLPs which comprises M protein from a BRSV, a chimeric RSV F and/or G protein and optionally HA or NA protein derived from an influenza virus, wherein the HA protein is fused to the transmembrane domain and cytoplasmic tail of RSV F and/or G protein.

In one embodiment, the disclosure comprises an immunogenic formulation comprising at least one effective dose of a modified or mutated RSV F protein. In another embodiment, the disclosure comprises an immunogenic formulation comprising at least one effective dose of an RSV F micelle comprising a modified or mutated RSV F protein. In yet another embodiment, the disclosure comprises an immunogenic formulation comprising at least one effective dose of a VLP comprising a modified or mutated RSV F protein as described above.

The compositions disclosed herein may be combined with pharmaceutically acceptable carrier or excipient. Pharmaceutically acceptable carriers include but are not limited to saline, buffered saline, dextrose, water, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition). The formulation should suit the mode of administration. In a preferred embodiment, the formulation is suitable for administration to humans, preferably is sterile, non-particulate and/or non-pyrogenic.

The composition, if desired, can also contain minor amounts of wetting or emulsifying agents, or pH buffering agents. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

The disclosure also provides for a pharmaceutical pack or kit comprising one or more containers filled with one or more of the ingredients of the vaccine formulations. In a preferred embodiment, the kit comprises two containers, one containing a modified or mutated RSV F protein in nanoparticle form, an RSV F micelle comprising a modified or mutated RSV F protein, or a VLP comprising a modified or mutated RSV F protein, as an RSV component, and the other containing an influenza component, such as an influenza VLP. Each component may be formulated with an adjuvant or may be formulated with a different buffer. Associated with such container(s) can be a notice in the form prescribed by a governmental agency regulating the manufacture, use or sale of pharmaceuticals or biological products, which notice reflects approval by the agency of manufacture, use or sale for human administration.

In some aspects, the compositions are provided in separate container, such as vials, and combined in a single container immediately before administration. In other aspects the compositions are prepared separately, combined, and then stored in the same container prior to use.

The disclosure also provides that the formulation be packaged in a hermetically sealed container such as an ampoule or sachette indicating the quantity of composition. In one embodiment, the composition is supplied as a liquid, in another embodiment, as a dry sterilized lyophilized powder or water free concentrate in a hermetically sealed container and can be reconstituted, e.g., with water or saline to the appropriate concentration for administration to a subject.

In an alternative embodiment, the composition is supplied in liquid form in a hermetically sealed container indicating the quantity and concentration of the composition. Preferably, the liquid form of the composition is supplied in a hermetically sealed container at least about 50 μg/ml, more preferably at least about 100 μg/ml, at least about 200 μg/ml, at least 500 μg/ml, or at least 1 mg/ml.

As an example, chimeric RSV VLPs comprising a modified or mutated RSV F protein of the disclosure are administered in an effective amount or quantity (as defined above) sufficient to stimulate an immune response, each a response against one or more strains of RSV. Administration of the modified or mutated RSV F protein, an RSV F micelle comprising a modified or mutated RSV F protein, or VLP of the disclosure elicits immunity against RSV. Typically, the dose can be adjusted within this range based on, e.g., age, physical condition, body weight, sex, diet, time of administration, and other clinical factors. The prophylactic vaccine formulation is systemically administered, e.g., by subcutaneous or intramuscular injection using a needle and syringe, or a needle-less injection device. Alternatively, the vaccine formulation is administered intranasally, either by drops, large particle aerosol (greater than about 10 microns), or spray into the upper respiratory tract. While any of the above routes of delivery results in an immune response, intranasal administration confers the added benefit of eliciting mucosal immunity at the site of entry of many viruses, including RSV and influenza.

Thus, the disclosure also comprises a method of formulating a vaccine or antigenic composition that induces immunity to an infection or at least one disease symptom thereof to a mammal, comprising adding to the formulation an effective dose of a modified or mutated RSV F protein, an RSV F micelle comprising a modified or mutated RSV F protein, or a VLP comprising a modified or mutated RSV F protein. In one embodiment, the infection is an RSV infection.

While stimulation of immunity with a single dose is possible, additional dosages can be administered, by the same or different route, to achieve the desired effect. In neonates and infants, for example, multiple administrations may be required to elicit sufficient levels of immunity. Administration can continue at intervals throughout childhood, as necessary to maintain sufficient levels of protection against infections, e.g. RSV infection. Similarly, adults who are particularly susceptible to repeated or serious infections, such as, for example, health care workers, day care workers, family members of young children, the elderly, and individuals with compromised cardiopulmonary function may require multiple immunizations to establish and/or maintain protective immune responses. Levels of induced immunity can be monitored, for example, by measuring amounts of neutralizing secretory and serum antibodies, and dosages adjusted or vaccinations repeated as necessary to elicit and maintain desired levels of protection.

### Administering the Compositions

Methods of administering the combination compositions disclosed herein include, but are not limited to, parenteral administration (e.g., intradermal, intramuscular, intravenous and subcutaneous), epidural, and mucosal (e.g., intranasal and oral or pulmonary routes or by suppositories). Administration is preferably intramuscular. The compositions may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. In some embodiments, intranasal or other mucosal routes of administration of a composition of the disclosure may induce an antibody or other immune response that is substantially higher than other routes of administration. Administration may be systemic or local.

In some embodiments, the compositions may be administered simultaneously via the same needle (i.e co-administered) wherein the RSV F and influenza components have been mixed together. In other embodiments, the influenza and RSV F components may administered sequentially (i.e., via separate administrations of each component over a short period of time; for example, the components may be administered about 1 minute apart, about 2 minutes apart, or about 5 minutes apart. In some embodiments the administrations may be spaced throughout the day. In some embodiments, the RSV and influenza components can be administered to the same area. In some embodiments, the RSV F and influenza components can be administered sequentially on different body parts; for example, the components may be administered sequentially on opposite arms, an arm and buttock, or different buttock cheeks.

In yet another embodiment, the vaccine and/or immunogenic formulation is administered in such a manner as to target mucosal tissues in order to elicit an immune response at the site of immunization. For example, mucosal tissues such as gut associated lymphoid tissue (GALT) can be targeted for immunization by using oral administration of compositions which contain adjuvants with particular mucosal targeting properties. Additional mucosal tissues can also be targeted, such as nasopharyngeal lymphoid tissue (NALT) and bronchial-associated lymphoid tissue (BALT).

Vaccines and/or immunogenic formulations of the disclosure may also be administered on a dosage schedule, for example, an initial administration of the vaccine composition with subsequent booster administrations. In particular embodiments, a second dose of the composition is administered anywhere from two weeks to one year, preferably from about 1, about 2, about 3, about 4, about 5 to about 6 months, after the initial administration. Additionally, a third dose may be administered after the second dose and from about three months to about two years, or even longer, preferably about 4, about 5, or about 6 months, or about 7 months to about one year after the initial administration. The third dose may be optionally administered when no or low levels of specific immunoglobulins are detected in the serum and/or urine or mucosal secretions of the subject after the second dose. In a preferred embodiment, a second dose is administered about one month after the first administration and a third dose is administered about six months after the first administration. In another embodiment, the second dose is administered about six months after the first administration. In another embodiment, the compositions of the disclosure can be administered as part of a combination therapy. For example, compositions of the disclosure can be formulated with other immunogenic compositions, antivirals and/or antibiotics.

The dosage of the pharmaceutical composition can be determined readily by the skilled artisan, for example, by first identifying doses effective to elicit a prophylactic or therapeutic immune response, e.g., by measuring the serum titer of virus specific immunoglobulins or by measuring the inhibitory ratio of antibodies in serum samples, or urine samples, or mucosal secretions. The dosages can be determined from animal studies. A non-limiting list of animals used to study the efficacy of vaccines include the guinea pig, hamster, ferrets, chinchilla, mouse and cotton rat. Most animals are not natural hosts to infectious agents but can still serve in studies of various aspects of the disease. For example, any of the above animals can be dosed with a vaccine candidate, e.g. modified or mutated RSV F proteins, an RSV F micelle comprising a modified or mutated RSV F protein, or VLPs of the disclosure, to partially characterize the immune response induced, and/or to determine if any neutralizing antibodies have been produced. For example, many studies have been conducted in the mouse model because mice are small size and their low cost allows researchers to conduct studies on a larger scale.

In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

In some aspects, the RSV and influenza compositions may be administered to children, young adults, women of child bearing age, and the elderly. In some embodiments, the elderly patients may be 50, 55, 60, 65, 70, 75, 80, 85, 90, 95, 100, or 105 years old. In some embodiments, the elderly patients are between 50 and 100 years old. In some embodiments, the elderly patients are between about 65 to about 85 years old.

### Methods of Stimulating an Immune Response

The modified or mutated RSV F proteins, the RSV F micelles comprising a modified or mutated RSV F protein, and the RSV and influenza VLPs of the disclosure, are useful for preparing compositions that stimulate an immune response that confers immunity or substantial immunity to infectious agents. Both mucosal and cellular immunity may contribute to immunity to infectious agents and disease. Antibodies secreted locally in the upper respiratory tract are a major factor in resistance to natural infection. Secretory immunoglobulin A (sIgA) is involved in the protection of the upper respiratory tract and serum IgG in protection of the lower respiratory tract. The immune response induced by an infection protects against reinfection with the same virus or an antigenically similar viral strain. For example, RSV undergoes frequent and unpredictable changes; therefore, after natural infection, the effective period of protection provided by the host's immunity may only be effective for a few years against the new strains of virus circulating in the community.

Thus, the disclosure encompasses a method of inducing immunity to infections or at least one disease symptom thereof in a subject, comprising administering at least one effective dose of a modified or mutated RSV F protein, an RSV F micelle comprising a modified or mutated RSV F protein, or a VLP comprising a modified or mutated RSV F protein. In one embodiment, the method comprises administering VLPs comprising a modified or mutated RSV F protein and at least one additional protein. In another embodiment, the method comprises administering VLPs further comprising an RSV M protein, for example, a BRSV M protein. In another embodiment, the method comprises administering VLPs further comprising a RSV N protein. In another embodiment, the method comprises administering VLPs further comprising a RSV G protein. In another embodiment, the method comprises administering VLPs further comprising a RSV SH protein. In another embodiment, the method comprises administering VLPs further comprising F and/or G protein from HRSV group A and/or group B. In another embodiment, the method comprises administering VLPs comprising M protein from BRSV and a chimeric RSV F and/or G protein or MMTV envelope protein, for example, HA or NA protein derived from an influenza virus, wherein the HA and/or NA protein is fused to the transmembrane domain and cytoplasmic tail of the RSV F and/or G protein or MMTV envelope protein. In another embodiment, the method comprises administering VLPs comprising M protein from BRSV and a chimeric RSV F and/or G protein and optionally HA or NA protein derived from an influenza virus, wherein the HA or NA protein is fused to the transmembrane domain and cytoplasmic tail of RSV F and/or G protein. In another embodiment, the subject is a mammal. In another embodiment, the mammal is a human. In another embodiment, RSV VLPs are formulated with an adjuvant or immune stimulator.

In one embodiment, the disclosure comprises a method to induce immunity to RSV infection or at least one disease symptom thereof in a subject, comprising administering at least one effective dose of a modified or mutated RSV F protein. In another embodiment, the disclosure comprises a method to induce immunity to RSV infection or at least one disease symptom thereof in a subject, comprising administering at least one effective dose of an RSV F micelle comprising a modified or mutated RSV F protein. In yet another embodiment, the disclosure comprises a method to induce immunity to RSV infection or at least one disease symptom thereof in a subject, comprising administering at least one effective dose of RSV VLPs, wherein the VLPs comprise a modified or mutated RSV F protein, M, G, SH, and/or N proteins. In another embodiment, a method of inducing immunity to RSV infection or at least one symptom thereof in a subject, comprises administering at least one effective dose of a RSV VLPs, wherein the VLPs consists essentially of BRSV M (including chimeric M), and RSV F, G, and/or N proteins. The VLPs may comprise additional RSV proteins and/or protein contaminates in negligible concentrations. In another embodiment, a method of inducing immunity to RSV infection or at least one symptom thereof in a subject, comprises administering at least one effective dose of a RSV VLPs, wherein the VLPs consists of BRSV M (including chimeric M), RSV G and/or F. In another embodiment, a method of inducing immunity to RSV infection or at least one disease symptom in a subject, comprises administering at least one effective dose of a RSV VLPs comprising RSV proteins, wherein the RSV proteins consist of BRSV M (including chimeric M), F, G, and/or N proteins, including chimeric F, G, and/or N proteins. These VLPs contain BRSV M (including chimeric M), RSV F, G, and/or N proteins and may contain additional cellular constituents such as cellular proteins, baculovirus proteins, lipids, carbohydrates etc., but do not contain additional RSV proteins (other than fragments of BRSV M (including chimeric M), BRSV/RSV F, G, and/or N proteins. In another embodiment, the subject is a vertebrate. In one embodiment the vertebrate is a mammal. In another embodiment, the mammal is a human. In another embodiment, the method comprises inducing immunity to RSV infection or at least one disease symptom by administering the formulation in one dose. In another embodiment, the method comprises inducing immunity to RSV infection or at least one disease symptom by administering the formulation in multiple doses.

The disclosure also encompasses inducing immunity to an infection, or at least one symptom thereof, in a subject caused by an infectious agent, comprising administering at least one effective dose of a modified or mutated RSV F protein, an RSV F micelle comprising a modified or mutated RSV F protein, or a VLP comprising a modified or mutated RSV F protein. In one embodiment, the method comprises administering VLPs comprising a modified or mutated RSV F protein and at least one protein from a heterologous infectious agent. In one embodiment, the method comprises administering VLPs comprising a modified or mutated RSV F protein and at least one protein from the same or a heterologous infectious agent. In another embodiment, the protein from the heterologous infectious agent is a viral protein. In another embodiment, the protein from the infectious agent is an envelope associated protein. In another embodiment, the protein from the infectious agent is expressed on the surface of VLPs. In another embodiment, the protein from the infectious agent comprises an epitope that will generate a protective immune response in a vertebrate. In another embodiment, the protein from the infectious agent can associate with RSV M protein such as BRSV M protein, RSV F, G and/or N protein. In another embodiment, the protein from the infectious agent is fused to a RSV protein such as a BRSV M protein, RSV F, G and/or N protein. In another embodiment, only a portion of a protein from the infectious agent is fused to a RSV protein such as a BRSV M protein, RSV F, G and/or N protein. In another embodiment, only a portion of a protein from the infectious agent is fused to a portion of a RSV protein such as a BRSV M protein, RSV F, G and/or N protein. In another embodiment, the portion of the protein from the infectious agent fused to the RSV protein is expressed on the surface of VLPs. In other embodiment, the RSV protein, or portion thereof, fused to the protein from the infectious agent associates with the RSV M protein. In other embodiment, the RSV protein, or portion thereof, is derived from RSV F, G, N and/or P. In another embodiment, the chimeric VLPs further comprise N and/or P protein from RSV. In another embodiment, the chimeric VLPs comprise more than one protein from the same and/or a heterologous infectious agent. In another embodiment, the chimeric VLPs comprise more than one infectious agent protein, thus creating a multivalent VLP.

Compositions of the disclosure can induce substantial immunity in a vertebrate (e.g. a human) when administered to the vertebrate. The substantial immunity results from an immune response against compositions of the disclosure that protects or ameliorates infection or at least reduces a symptom of infection in the vertebrate. In some instances, if the vertebrate is infected, the infection will be asymptomatic. The response may not be a fully protective response. In this case, if the vertebrate is infected with an infectious agent, the vertebrate will experience reduced symptoms or a shorter duration of symptoms compared to a non-immunized vertebrate.

In one embodiment, the disclosure comprises a method of inducing substantial immunity to RSV virus infection or at least one disease symptom in a subject, comprising administering at least one effective dose of a modified or mutated RSV F protein, an RSV F micelle comprising a modified or mutated RSV F protein, or a VLP comprising a modified or mutated RSV F protein. In another embodiment, the disclosure comprises a method of vaccinating a mammal against RSV comprising administering to the mammal a protection-inducing amount of a modified or mutated RSV F protein, an RSV F micelle comprising a modified or mutated RSV F protein, or a VLP comprising a modified or mutated RSV F protein. In one embodiment, the method comprises administering VLPs further comprising an RSV M protein, such as BRSV M protein. In another embodiment, the method further comprises administering VLPs comprising RSV G protein, for example a HRSV G protein. In another embodiment, the method further comprises administering VLPs comprising the N protein from HRSV group A. In another embodiment, the method further comprises administering VLPs comprising the N protein from HRSV group B. In another embodiment, the method comprises administering VLPs comprising chimeric M protein from BRSV and F and/or G protein derived from RSV wherein the F and/or G protein is fused to the transmembrane and cytoplasmic tail of the M protein. In another embodiment, the method comprises administering VLPs comprising M protein from BRSV and chimeric RSV F and/or G protein wherein the F and/or G protein is a fused to the transmembrane domain and cytoplasmic tail of influenza HA and/or NA protein. In another embodiment, the method comprises administering VLPs comprising M protein from BRSV and chimeric RSV F and/or G protein and optionally an influenza HA and/or NA protein wherein the F and/or G protein is a fused to the transmembrane domain and cytoplasmic tail of the HA protein. In another embodiment, the method comprises administering VLPs comprising M protein from BRSV and chimeric RSV F and/or G protein, and optionally an influenza HA and/or NA protein wherein the HA and/or NA protein is fused to the transmembrane domain and cytoplasmic tail of RSV F and/or G protein.

The disclosure also encompasses a method of inducing substantial immunity to an infection, or at least one disease symptom in a subject caused by an infectious agent, comprising administering at least one effective dose of a modified or mutated RSV F protein, an RSV F micelle comprising a modified or mutated RSV F protein, or a VLP comprising a modified or mutated RSV F protein. In one embodiment, the method comprises administering VLPs further comprising a RSV M protein, such as BRSV M protein, and at least one protein from another infectious agent. In one embodiment, the method comprises administering VLPs further comprising a BRSV M protein and at least one protein from the same or a heterologous infectious agent. In another embodiment, the protein from the infectious agent is a viral protein. In another embodiment, the protein from the infectious agent is an envelope associated protein. In another embodiment, the protein from the infectious agent is expressed on the surface of VLPs. In another embodiment, the protein from the infectious agent comprises an epitope that will generate a protective immune response in a vertebrate. In another embodiment, the protein from the infectious agent can associate with RSV M protein. In another embodiment, the protein from the infectious agent can associate with BRSV M protein. In another embodiment, the protein from the infectious agent is fused to a RSV protein. In another embodiment, only a portion of a protein from the infectious agent is fused to a RSV protein. In another embodiment, only a portion of a protein from the infectious agent is fused to a portion of a RSV protein. In another embodiment, the portion of the protein from the infectious agent fused to the RSV protein is expressed on the surface of VLPs. In other embodiment, the RSV protein, or portion thereof, fused to the protein from the infectious agent associates with the RSV M protein. In other embodiment, the RSV protein, or portion thereof, fused to the protein from the infectious agent associates with the BRSV M protein. In other embodiment, the RSV protein, or portion thereof, is derived from RSV F, G, N and/or P. In another embodiment, the VLPs further comprise N and/or P protein from RSV. In another embodiment, the VLPs comprise more than one protein from the infectious agent. In another embodiment, the VLPs comprise more than one infectious agent protein, thus creating a multivalent VLP.

In another embodiment, the disclosure comprises a method of inducing a protective antibody response to an infection or at least one symptom thereof in a subject, comprising administering at least one effective dose of a modified or mutated RSV F protein, an RSV F micelle comprising a modified or mutated RSV F protein, or a VLP comprising a modified or mutated RSV F protein as described above.

As used herein, an “antibody” is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. Antibodies exist as intact immunoglobulins or as a number of well-characterized fragments produced by digestion with various peptidases.

In one embodiment, the disclosure comprises a method of inducing a protective cellular response to RSV infection and to influenza infection, or at least one symptom of each disease in a subject, comprising administering at least one effective dose of a modified or mutated RSV F protein and an influenza component. In another embodiment, the disclosure comprises a method of inducing a protective cellular response to RSV infection or at least one disease symptom in a subject, comprising administering at least one effective dose an RSV F micelle comprising a modified or mutated RSV F protein. In yet another embodiment, the disclosure comprises a method of inducing a protective cellular response to RSV infection or at least one disease symptom in a subject, comprising administering at least one effective dose a VLP, wherein the VLP comprises a modified or mutated RSV F protein as described above. Cell-mediated immunity also plays a role in recovery from RSV infection and may prevent RSV-associated complications. RSV-specific cellular lymphocytes have been detected in the blood and the lower respiratory tract secretions of infected subjects. Cytolysis of RSV-infected cells is mediated by CTLs in concert with RSV-specific antibodies and complement. The primary cytotoxic response is detectable in blood after 6-14 days and disappears by day 21 in infected or vaccinated individuals (Ennis et al., 1981). Cell-mediated immunity may also play a role in recovery from RSV infection and may prevent RSV-associated complications. RSV-specific cellular lymphocytes have been detected in the blood and the lower respiratory tract secretions of infected subjects.

As mentioned above, the immunogenic compositions of the disclosure prevent or reduce at least one symptom of RSV infection in a subject. Symptoms of RSV are well known in the art. They include rhinorrhea, sore throat, headache, hoarseness, cough, sputum, fever, rales, wheezing, and dyspnea. Thus, the method of the disclosure comprises the prevention or reduction of at least one symptom associated with RSV infection. A reduction in a symptom may be determined subjectively or objectively, e.g., self assessment by a subject, by a clinician's assessment or by conducting an appropriate assay or measurement (e.g. body temperature), including, e.g., a quality of life assessment, a slowed progression of a RSV infection or additional symptoms, a reduced severity of a RSV symptoms or a suitable assays (e.g. antibody titer and/or T-cell activation assay). The objective assessment comprises both animal and human assessments.

This disclosure is further illustrated by the following examples that should not be construed as limiting. The contents of all references, patents and published patent applications cited throughout this application, as well as the Figures and the Sequence Listing, are incorporated herein by reference for all purposes.

### EXAMPLES

### Example 1

### Mice Study

80 Balb/c mice age 6-8 weeks old were injected with candidate vaccines according to the protocol described in Table 1.

TABLE 1
Study Design for Mice Trial with Trivalent
Flu Component and RSV F component

| | | | Tri- | RSV F | | |
|---|---|---|---|---|---|---|
| | | | Flu | Antigen | Immuni- | |
| | | Mice | Dose | Dose | zation | |
| Group | Antigen | (N) | (μg) | (μg) | Days | Animal No. |
| 1 | Trivalent | 8 | 3 | 3 | 0, 21 | 12-0123-01 to |
| | Flu + | | | | | 12-0123-08 |
| | RSV | | | | | |
| 2 | Trivalent | 8 | 9 | 9 | 0, 21 | 12-0123-09 to |
| | Flu + | | | | | 12-0123-16 |
| | RSV | | | | | |
| 3 | Flu + | 8 | 3 | — | 0, 21 | 12-0123-17 to |
| | Buffer 1 | | | | | 12-0123-24 |
| | (Flu) | | | | | |
| 4 | Flu + | 8 | 9 | — | 0, 21 | 12-0123-25 to |
| | Buffer 1 | | | | | 12-0123-32 |
| | (Flu) | | | | | |
| 5 | RSV + | 8 | — | 3 | 0, 21 | 12-0123-33 to |
| | Buffer 2 | | | | | 12-0123-40 |
| | (RSV) | | | | | |
| 6 | RSV + | 8 | — | 9 | 0, 21 | 12-0123-41 to |
| | Buffer 2 | | | | | 12-0123-48 |
| | (RSV) | | | | | |
| 7 | Flu + | 8 | 3 | — | 0, 21 | 12-0123-49 to |
| | Buffer 2 | | | | | 12-0123-56 |
| | (RSV) | | | | | |
| 8 | Flu + | 8 | 9 | — | 0, 21 | 12-0123-57 to |
| | Buffer 2 | | | | | 12-0123-64 |
| | (RSV) | | | | | |
| 9 | RSV + | 8 | — | 3 | 0, 21 | 12-0123-65 to |
| | Buffer 1 | | | | | 12-0123-72 |
| | (Flu) | | | | | |
| 10 | RSV + | 8 | — | 9 | 0, 21 | 12-0123-73 to |
| | Buffer 1 | | | | | 12-0123-80 |
| | (Flu) | | | | | |

Buffer 1 “Flu Buffer” contained 25 mM sodium phosphate buffer, pH 7.2, 500 mM sodium chloride, 0.3 mM CaCl2 and 0.01% w/v PS80. Buffer 2 “RSV Buffer” contained 25 mM phosphate, 0.15 M NaCl, 0.01% (W/V) PS80, (w/v) Histidine pH 6.2.

A trivalent composition containing three influenza components was used to stimulate an anti-influenza response. The trivalent composition contained three VLPs of the following strains: A-Perth H3N2 S205 (Victoria), A-Cal H1N1, and B-Wisconsin. Each VLP contains both the HA and NA proteins from the recited strain. The M1 protein for all three strains was derived from A/Indonesia/5/05. The lot numbers Lot Number: 75511013, 75511008A, 5511009. The influenza component is 0.25% BPL treated, 0.2 μm filtered Single Radial Immuno diffusion (SRID). The respective HA levels were: 354, 626, 280 μg HA/ml. The component was stored in Buffer: 25 mM Phosphate, pH 7.2/0.5M NaCl/0.01% PS-80/300 μM CaCl₂ at temp: 2-8° C. The RSV F component (SEQ ID NO:8; prepared and purified as described in U.S. Ser. No. 13/269,107; Lot Number: 683.15p) was stored in buffer: 25 mM Sodium Phosphate, 0.15M NaCl, 0.01% (W/V) PS-80, 1% (W/V) Histidine pH 6.2 at temp: 2-8° C.

The RSV and trivalent influenza components were each administered in Buffer 1 “Flu” Buffer and Buffer 2, the “RSV” buffer. Buffer 1 contained 25 mM sodium phosphate buffer, pH 7.2, 500 mM sodium chloride, 0.3 mM CaCl2 and 0.01% w/v PS80. Buffer 2 “RSV Buffer” contained 25 mM phosphate, 0.15 M NaCl, 0.01% (W/V) PS80, (w/v) Histidine pH 6.2. The mice were injected at Days 0 and 21. Blood samples were taken from the mice at Days 0, 21, and 35. For Groups 1 and 2 in Table 1, the RSV and trivalent influenza components were combined prior to injection.

### Example 2

### Characterization of RSV F Antibodies

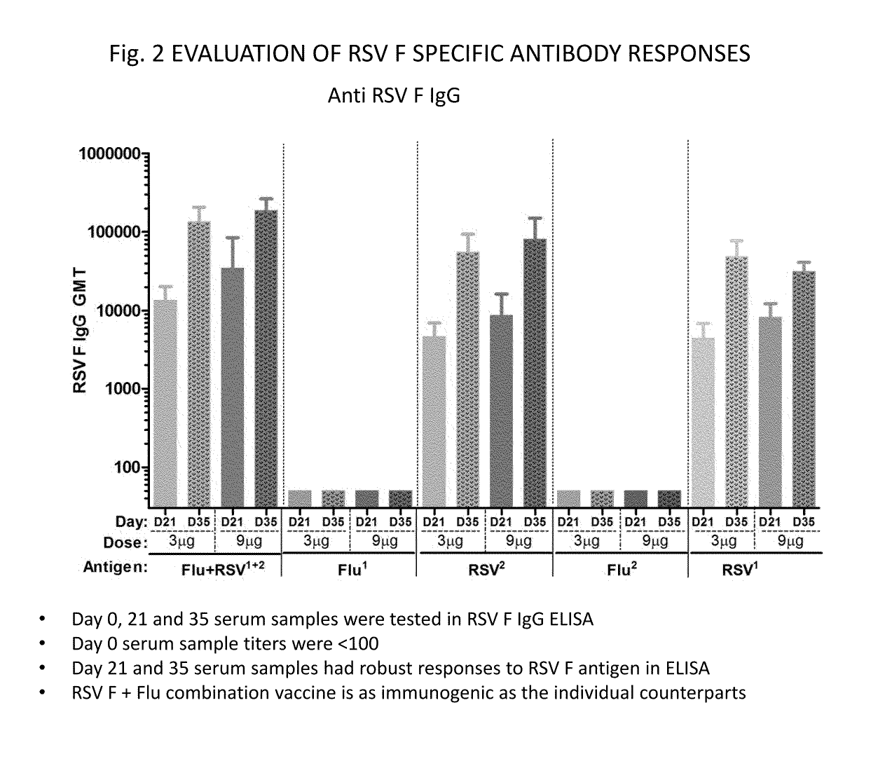

Mice were administered combination compositions as described in Example 1. FIG. 2 shows the anti-RSV F response obtained as measured by ELISA assay. Day 0 titers were <100 (not shown). As expected the flu component alone did not induce an anti-RSV F response. Administering the RSV component alone resulted in a robust anti-RSV F response. Robust responses were obtained with Buffer 1 and Buffer 2 and at doses of 3 μg and 9 μg both at Day 35 (D35) and Day 21 (D21). Day 35 titers were higher. Remarkably, when the trivalent influenza component was combined with the RSV component, an elevated anti-RSV F response was achieved.

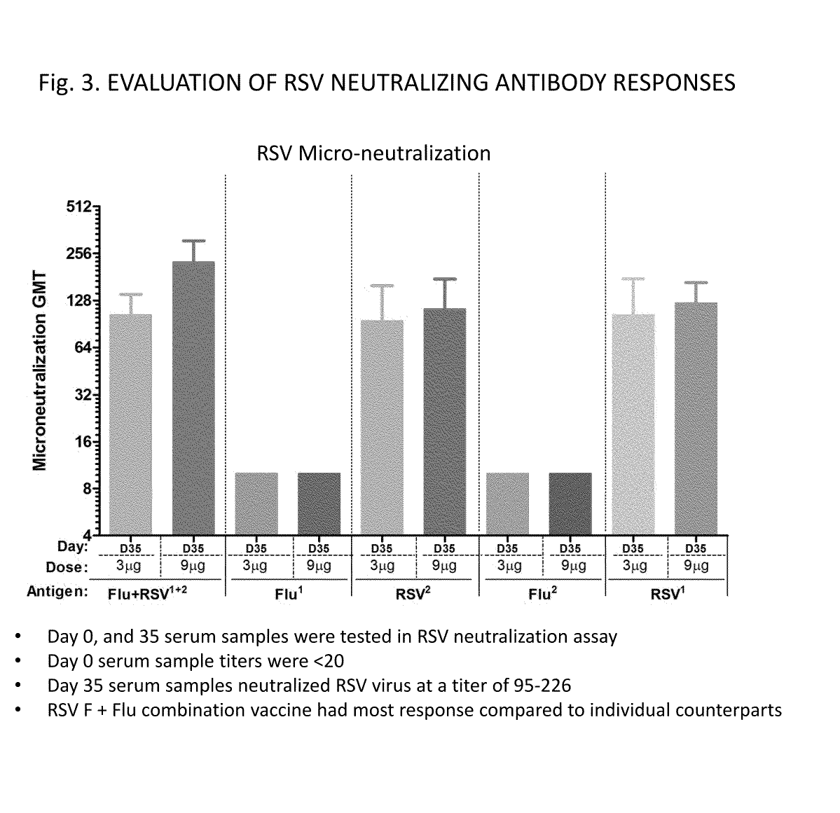

Similar data were achieved when the immune response was assessed to determine production of neutralizing antibodies. FIG. 3 shows neutralizing antibodies obtained in the trial described in Example 1. Neutralizing antibodies were measured at 35 days for each sample. Day 0 serum sample titers were <20 (not shown). Neutralizing anti-RSV response ranged from 95-226.

### Example 3

### Characterization of RSV F Palivizumab-Competitive Antibodies

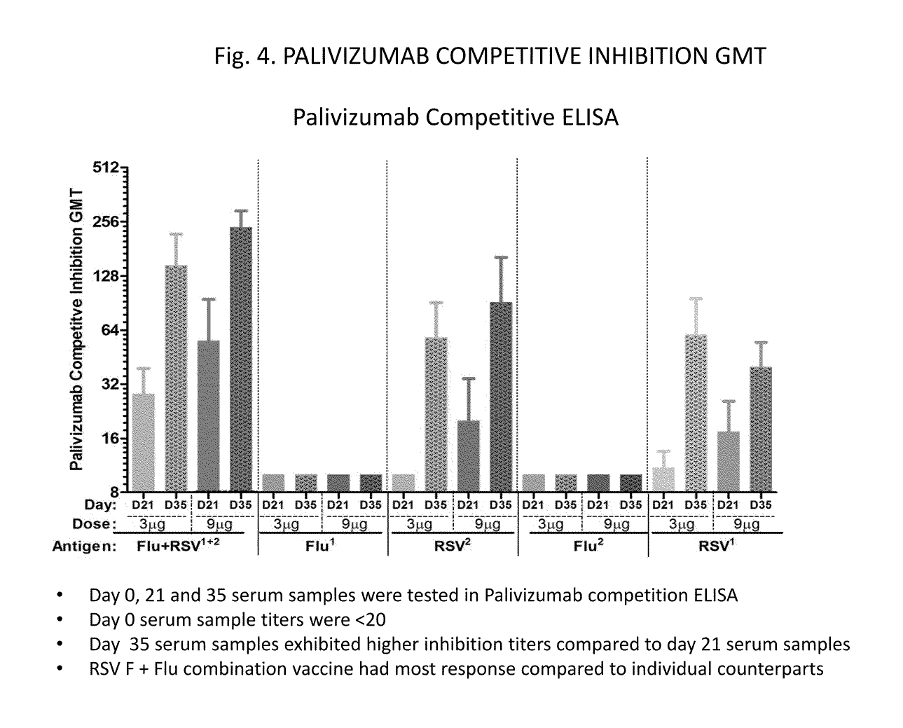

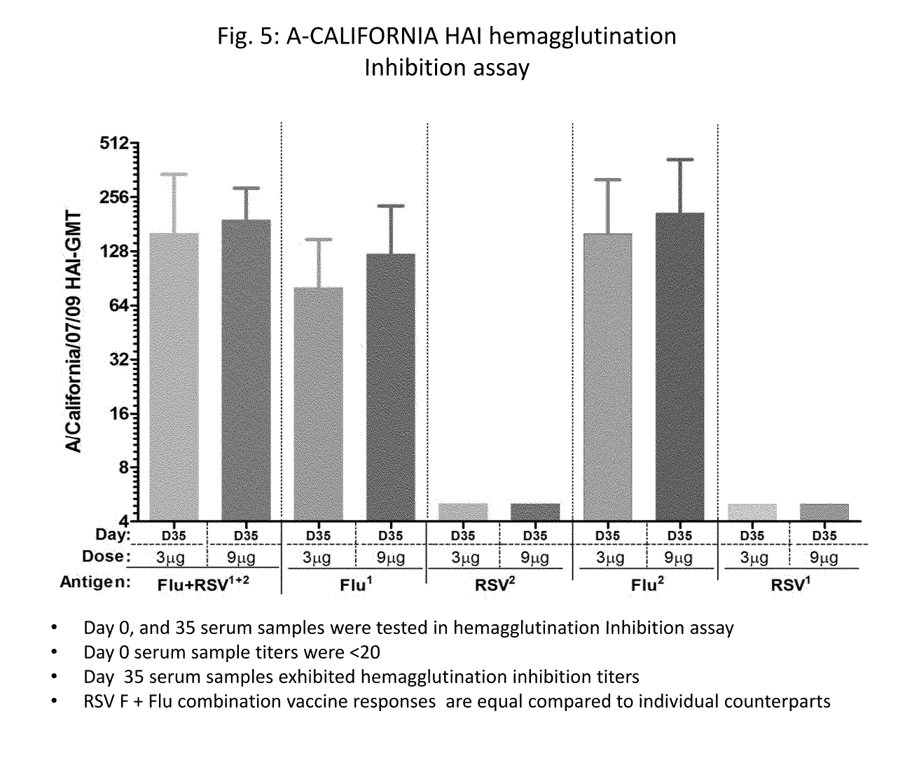

Palivizumab (Synagis™) is a monoclonal antibody that binds and neutralizes RSV viruses in humans. Palivizumab binds to an epitope on RSV F (SEQ ID NO:35). Advantageously, the RSV component stimulates an immune response against the same epitope. FIG. 4 is a palivizumab-competitive ELISA which shows that the antibody response induced by the combination composition binds to the same epitope recognized by Palivizumab. Day 0 serum titers were <20 (not shown). At Day 21 and Day 35 robust responses against the epitope were obtained with Buffer 1 and Buffer 2 and at doses of 3 μg and 9 μg. A similar response was obtained when the three flu components and RSV component were co-administered.

### Example 4

### Characterization of Anti-Influenza Response

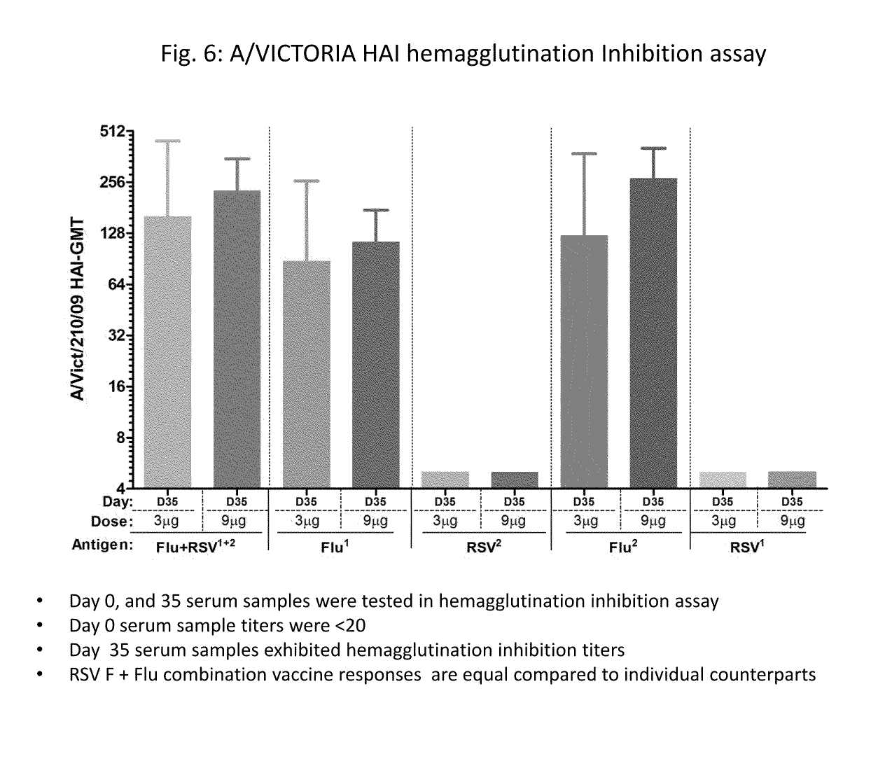

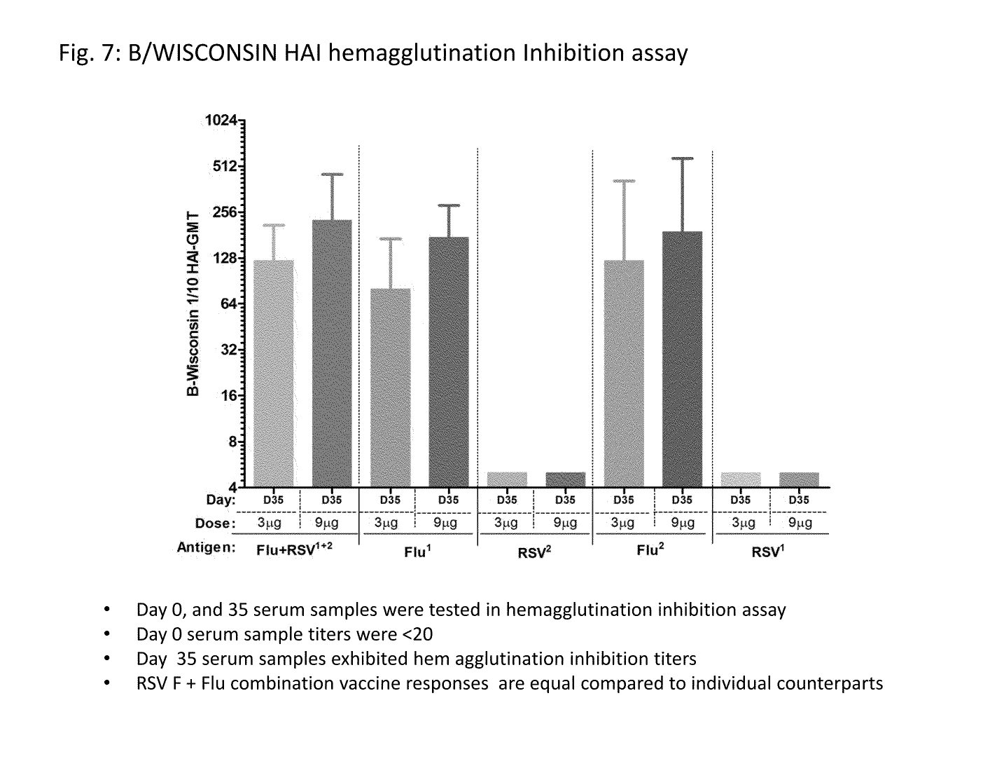

Mice were administered the vaccine compositions as described in Example 1. Hemagglutination Inhibition assays were performed to determine hemagglutination inhibition for each of the influenza components. As FIGS. 5-7 demonstrate, substantial inhibition was achieved for all three strains. FIG. 5 shows the A-California strain results. FIG. 6 shows the A/Victoria strain results. FIG. 7 shows the B/Wisconsin strain results. Day 0 serum titers were <20 (not shown). At Day 35 robust hemagglutination inhibition titers were obtained with Buffer 1 and Buffer 2 and at doses of 3 μg and 9 μg. The combination composition, containing the trivalent flu composition, (with three influenza components in VLP form) and the RSV component, induced better responses than the trivalent flu composition alone.

### Example 5

### Human Study

A clinical trial of RSV F sequential administration with influenza immunogenic compositions in elderly human population was conducted. For this trial, 220 human subjects were randomized to receive one dose of 60 or 90 μg of RSV F protein with or without 1200 μg of AlPO₄ adjuvant; or placebo. All subjects also received influenza vaccine (TIV), to mimic sequential administration likely to occur in use. Administration was conducted in sequential administrations via distinct intramuscular injections of RSV F and TIV doses to opposing arms of the same subject. The protocol and study documents were reviewed and approved by an appropriately constituted institutional review board; all subjects gave written informed consent. The experimental design was randomized, age-stratified, and observer-blinded. Operators for assays were blinded to subject treatments. A summary of the experimental treatments can be seen below:

TABLE 2
Human subject demography and experimental treatments.

| Group | E | A | B | C | D |
|---|---|---|---|---|---|
| RSV F | 0 | 60 ug | 60 ug | 90 ug | 90 ug |
| | (placebo) | | | | |
| AlPO₄ | No | Yes | No | Yes | No |
| N | 60 | 40 | 40 | 40 | 40 |
| Age (yrs) | | | | | |
| Mean | 69.1 | 69.1 | 67.7 | 68.0 | 68.7 |
| Median | 68.0 | 68.0 | 67.0 | 68.0 | 68.0 |
| % ≧75 | 15 | 15 | 15 | 15 | 15 |
| Male/female | 37/63% | 45/55% | 40/60% | 53/47% | 42/58% |
| Mean BMI | 27.4 | 28.5 | 27.7 | 29.6 | 27.6 |

Hemagglutination Inhibition assays were performed to determine hemagglutination inhibition for each of the influenza components. Influenza hemagglutination-inhibition (HAI) assays were performed at Novavax essentially following the established World Health Organization method. TIV responses as measured by post-immunization HAI seroconversions and GMTs, were entirely unaffected by RSV F sequential administration.

### Example 6

### Characterization of RSV F Antibodies in Human Study

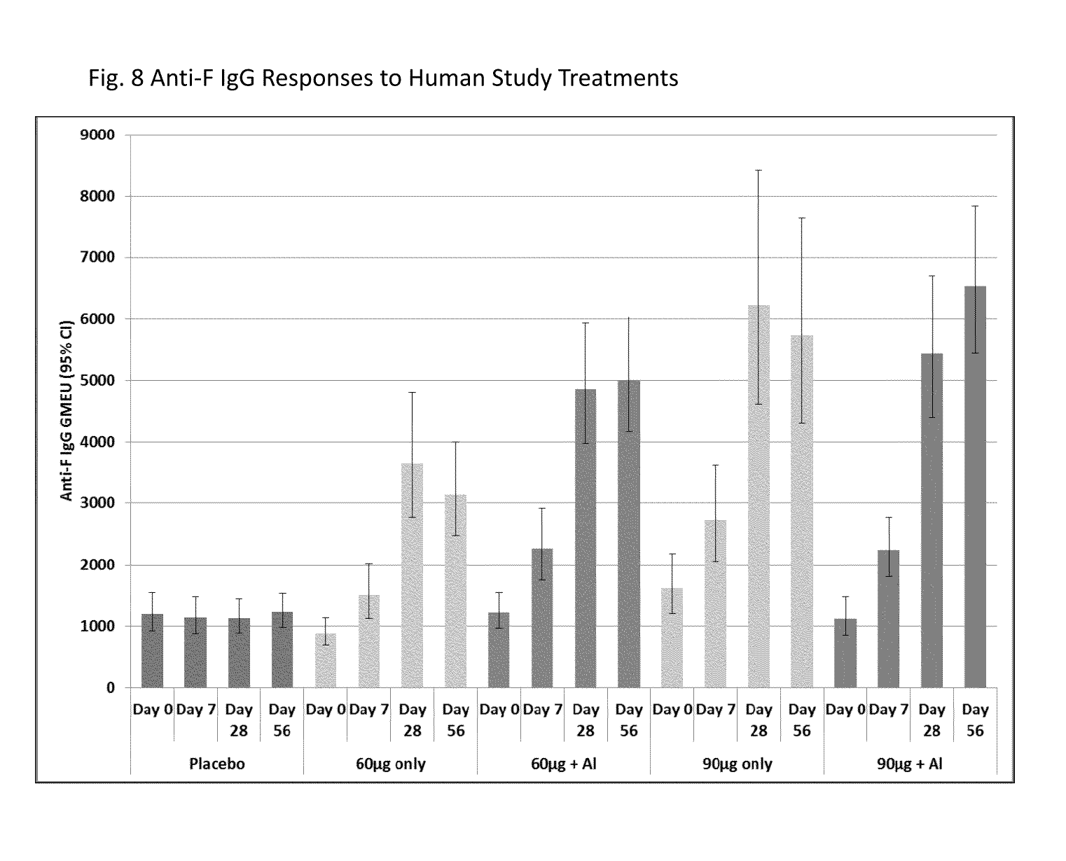

Human subjects were sequentially administered RSV F and TIV compositions as described in Example 5. FIG. 8 shows the anti-RSV F response obtained as measured by ELISA assay using protocols described in (Falsey A R, et al. Vaccine 2013; 31:524) As expected the placebo treatment alone did not induce an anti-RSV F response. Administering the RSV F component resulted in a robust anti-RSV F response. Levels of serum anti-F IgG rose 3.1 to 5.6 fold, with best responses in recipients of 90 μg+Al treatments. Serologic response rates in the Al-adjuvanted groups were 89-92%.

### Example 7

### Characterization of RSV F Palivizumab-Competitive Antibodies in Human Study

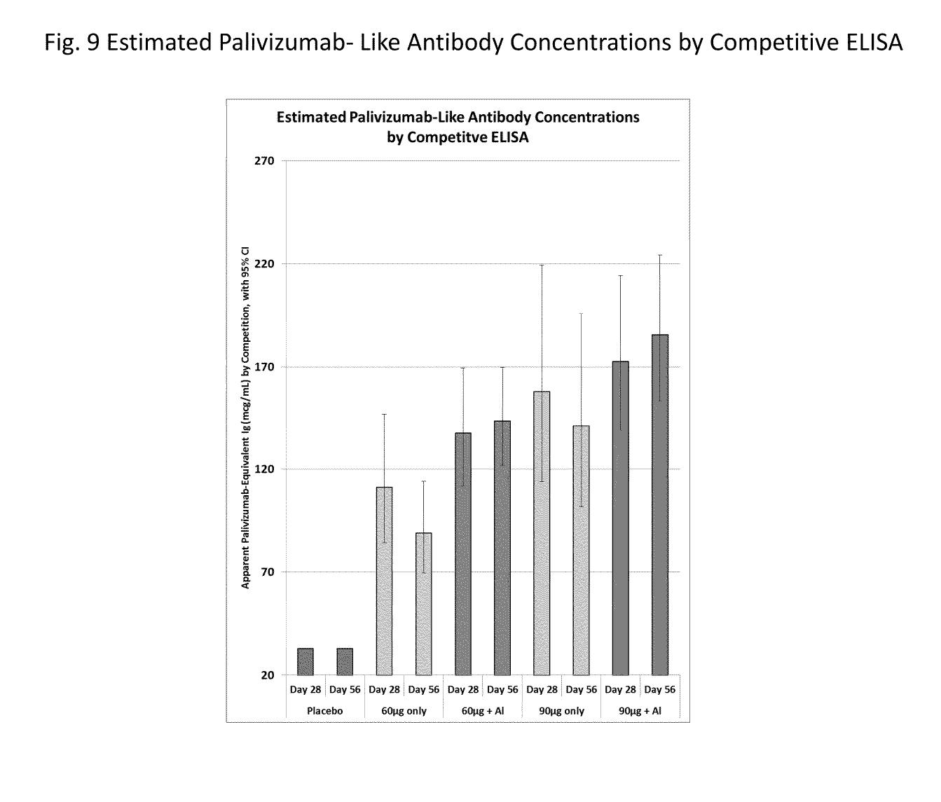

Palivizumab (Synagis™) is a monoclonal antibody that binds and neutralizes RSV viruses in humans. Palivizumab binds to an epitope on RSV F (SEQ ID NO:35). Advantageously, the RSV component stimulates an immune response against the same epitope. FIG. 9 contains the results of a palivizumab-competitive ELISA which shows that the antibody response induced by the sequential administration binds to the same epitope recognized by Palivizumab. At Day 28 and Day 56 robust responses against the epitope were obtained at both the 60 ug and 90 ug treatments with and without Alum. Antibodies competing with palivizumab rose from undetectable at day 0 to 85-185 mg/ml in experimental RSV treatments; response rates were 74-78% without Al and 97.4% with adjuvant.

### Example 8

### Characterization of Antibodies Against Antigenic Site II in Human Study

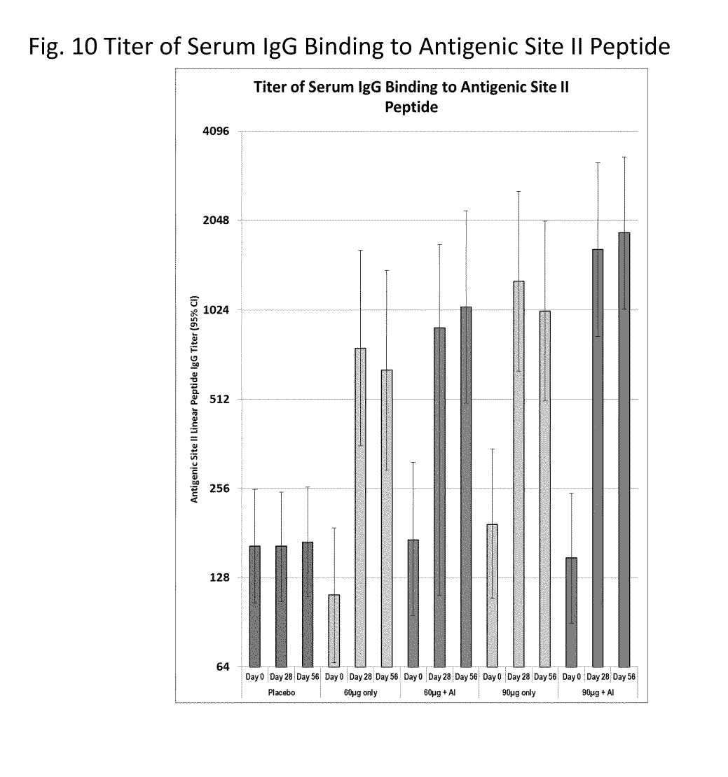

Human subjects were sequentially administered RSV F and TIV compositions as described in Example 5. As FIG. 10 demonstrates, substantial immunogenic responses against antigenic site II were achieved for all non-placebo treatments. Titers of IgG reactive with antigenic site II peptide rose 5.3 to 12.5-fold with the highest titers belonging to the 90 ug+Al treatment. These results demonstrate that the compositions induce immune responses that do not suffer from antigen interference.

### Example 9

### Key Safety Outcomes of Human RSV F and TIV Study

Human subjects were sequentially administered RSV F and TIV compositions as described in Example 5. Safety was assessed using reactogenicity diaries, safety laboratory tests, and open-ended queries concerning changes in health. Mean ages in the treatment groups were 67.7 to 69.1 years; 15% were ≧75 y.o. The majority of subjects were Caucasian and men comprised 43% overall; 99% of subjects provided data through Day 56. Among placebo recipients 70% reported at least 1 adverse event (AE), compared with 58-75% of active vaccines in various groups. Transient injection site pain was 15-20% more frequent in active vaccines, but otherwise the vaccine safety profile differed little from placebo; the 1 serious AE occurred in the placebo group. A summary of the results is presented below:

TABLE 3
Human trial, key safety outcome data.

| Group | E | A | B | C | D |
|---|---|---|---|---|---|
| RSV F | 0 | 60 ug | 60 ug | 90 ug | 90 ug |
| | (placebo) | | | | |
| AlPO₄ | No | Yes | No | Yes | No |
| N | 60 | 40 | 40 | 40 | 40 |
| Completed | 59 | 40 | 39 | 40 | 40 |
| D56 | | | | | |
| Adverse | | | | | |
| Events* | | | | | |
| Any | 42 (70%) | 30 (75%) | 25 (63%) | 27 (68%) | 23 (58%) |
| Solicited | 28 (47%) | 22 (55%) | 12 (30%) | 21 (53%) | 19 (48%) |
| AEs | | | | | |
| Local Sol. | 14 (23%) | 17 (43%) | 9 (23%) | 17 (43%) | 15 (38%) |
| AEs | | | | | |
| Systemic | 22 (37%) | 12 (30%) | 6 (15%) | 16 (40%) | 10 (25%) |
| Sol. AEs | | | | | |
| Severe Sol. | 1 (2%) | 1 (3%) | 0 | 0 | 0 |
| AEs | | | | | |
| Unsolicited | 31 (52%) | 18 (45%) | 19 (48%) | 16 (40%) | 16 (40%) |
| AEs | | | | | |
| Severe & | 2 (3%) | 0 | 0 | 0 | 0 |
| related AEs | | | | | |
| Serious AEs | 1 (2%) | 0 | 0 | 0 | 0 |

These results demonstrate that the RSV F vaccine is compatible with TIV sequential administration, well-tolerated by elders, and elicits increases in antibodies with potentially protective specificities. Increased immunogenic responses were seen in all Aluminum phosphate adjuvant experimental treatments.

### Example 10

### Mouse Study for Quadrivalent Influenza (Q-Flu) and RSV F Combination Vaccine

A total of 90 female BALB/c mice (10 per group), 6-8 weeks old, were used in this study. All animals received two IM vaccinations on day 0 and 21 with a 6.0, 1.5 or 0.5 μg dose of RSV F or quadrivalent seasonal influenza VLP vaccine, or the combined RSV F and influenza VLP vaccine as described in Table 1. Quadrivalent influenza vaccine contained 1.5, 0.375 or 0.125 μg per strain (25% of each strain).

Blood sampling occurred on days 0, 21, 35 for immunogenicity assessments.

TABLE 4
Study Design

| | | | Quadrivalent | | |
|---|---|---|---|---|---|
| | | RSV F Antigen | Influenza* | Immuni- | Blood |
| | Mice | Dose (RSV F | VLP Dose | zation | Draw |
| Group | (N) | Content, μg) | (Total HA μg) | (Days) | (Days) |
| 1 | 10 | 6.0 | — | 0, 21 | −1, 21, 35 |
| 2 | 10 | 1.5 | — | 0, 21 | −1, 21, 35 |
| 3 | 10 | 0.5 | — | 0, 21 | −1, 21, 35 |
| 4 | 10 | — | 6.0 | 0, 21 | −1, 21, 35 |
| 5 | 10 | — | 1.5 | 0, 21 | −1, 21, 35 |
| 6 | 10 | — | 0.5 | 0, 21 | −1, 21, 35 |
| 7 | 10 | 6.0 | 6.0 | 0, 21 | −1, 21, 35 |
| 8 | 10 | 1.5 | 1.5 | 0, 21 | −1, 21, 35 |
| 9 | 10 | 0.5 | 0.5 | 0, 21 | −1, 21, 35 |

TABLE 5
RSV F and Influenza Strains in a Quadrivalent flu composition.

| | | | Conc. |
|---|---|---|---|
| Drug Substance | Source | Lot# | (HA μg/mL) |
| RSV | PD | B13D001 | 538.5 |
| A/Cal/04/09- H1N1 | PD | X13I001B | 741.0 |
| A/Victoria/361/11- H3N2 | PD | X13G002 | 479.1 |
| B/Brisbane/60/08 | PD | X13H003 | 350.5 |
| B/Mass/2/12 | PD | X13H001 | 412.7 |

Group 1, 2, and 3 were prepared in RSV F buffer: 25 mM phosphate, pH 6.2, 0.15 M NaCl, 0.01% (w/v) Polysorbate 80, 1% (w/v) histidine. Group 4, 5, and 6 were prepared in influenza buffer: 25 mM sodium phosphate, pH 7.2, 0.3M NaCl, 300 μM CaCl₂ and 0.01% (w/v) Polysorbate 80. Group 7, 8, and 9 were prepared in 50% influenza-50% RSV mixed buffer.

Immunological Methods

a) Anti-RSV F IgG ELISA

RSV F specific antibody titers were evaluated by enzyme linked immunosorbent assay (ELISA) in serum samples collected on days 0, 21 and 35. Briefly, NUNC MaxiSorp microtiter plates were coated with 2 μg/ml of RSV F protein and incubated overnight at 2-8° C. Unreacted surface was blocked with starting block (Pierce biological) for one hour at room temperature. Serial dilutions (5 fold, 1:100 to 1:390,625) of mice sera were prepared in duplicates and added to RSV F protein coated plates, incubated for two-hours at room temperature, and washed with phosphate buffered saline containing tween, PBS-T (Quality Biologicals). Following the addition of horseradish peroxidase conjugated goat anti-mouse-IgG (Southern Biotech), microtiter plates were incubated for 1-hour, washed three times with phosphate buffered saline containing tween, and the peroxidase substrate 3,3′,5,5′-tetramethylbenzidine (Sigma) was added to the plate to detect protein bound anti RSV F mouse IgG antibody. The color development was allowed to proceed for approximately 5-6 minutes. After addition of the TMB Stop Buffer (Scy Tek Laboratories) plates were read at 450 nm in SpectraMax plus plate readers (Molecular Devices). Data was analyzed using SoftMax pro software (Molecular Devices). A 4 PL curve was fitted to the data and titers were determined as the reciprocal value of the serum dilution that resulted in an OD450 of 1.0. Positive RSV F mouse sera was used as control. Pre-bleed mouse sera with a titer<100 served as the negative control.

b) Palivizumab-Competitive ELISA

Competitive binding of the RSV F mouse sera and biotin labeled Palivizumab (MedImmune LLC) to RSV F antigen was performed in 96 well microtiter plates. Palivizumab (10 mg/ml) was biotinylated with a biotin labeling kit (Pierce) as per manufacturer's instructions. Nunc MaxiSorb microtiter plates were coated with 2 μg/ml RSV-F antigen and incubated overnight at 2-8° C. Unreacted sites were then blocked with 1% milk at room temperature for one hour. Two-fold serial dilutions (from 1:20 to 1:1280) of mice sera were prepared in duplicate and spiked with 120 ng/ml of biotinylated palivizumab. Plates were incubated for two-hours at room temperature and washed with phosphate buffered saline containing tween (Quality Biologicals). Following the addition of streptavidin-conjugated horseradish peroxidase, (e-Bioscience) microtiter plates were incubated for 1-hour temp. After washing three times with phosphate buffered saline containing tween, the peroxidase substrate 3,3′,5,5′-Tetramethylbenzidine (Sigma) was added to the plate to detect antigen bound biotinylated palivizumab. After addition of the TMB Stop Buffer (Scy Tek Laboratories) plates were read at 450 nm in SpectraMax plus plate readers (Molecular Devices). Wells containing biotinylated palivizumab in buffer represented the un-competed and wells containing PBS alone without any biotin labeled palivizumab were used as negative controls in the assay. Positive RSV F mouse sera and pre-immune mouse sera were used as assay controls. Data were analyzed using SoftMax Pro software (Molecular Devices). The competed binding titers were expressed as the 50% inhibition titers. Percent inhibition titers were calculated for each serum dilution using the following formula: (OD_{palivizumab}−ODₛₐₘₚₗₑ/OD_{palivizumab})×100%.

A 4 PL curve was fitted to the data and titers were determined as the reciprocal value of the serum dilution that resulted in 50% inhibition of biotynilated palivizumab binding. In cases when the 50% inhibition could not be obtained, a titer of <20 was reported for the sample and a value of 10 used in calculating group GMTs.

c) Microneutralization

To determine whether the RSV F particle vaccine can elicit RSV neutralizing antibodies, sera samples from day 35 were assayed in a RSV-A Long strain neutralization assay. Two-fold serial dilutions of mice sera, starting at 1:20, were prepared in 96 well plates. An equal volume (50 μl) of virus (˜200 PFU) was added to the diluted serum and incubated for 1 hr at 36° C. 100 μl of freshly trypsinized HEp-2 cells (5×10⁵ cells/ml) in growth medium (L-15, 10% fetal bovine serum and 2 mM glutamine) was added to the virus/serum mixture and incubated for 6-7 days at 36° C. or until positive control (virus only) wells showed 100% cytopathic effect (CPE).

Cells were scored for CPE microscopically, before and/or after, fixing and staining with 0.25% crystal violet in 5% gluteraldehyde. Stained plates were air-dried and evaluated for CPE using a dissecting microscope. The last dilution that resulted in 100% inhibition of CPE formation was identified as the endpoint neutralizing antibody titer for that sample. Any sample resulting in a titer less than 20 was assigned a value of 10. The geometric mean from each group was calculated. Sheep RSV F serum with a titer of 6400 was used as positive control.

d) HAI Antibody Measurement

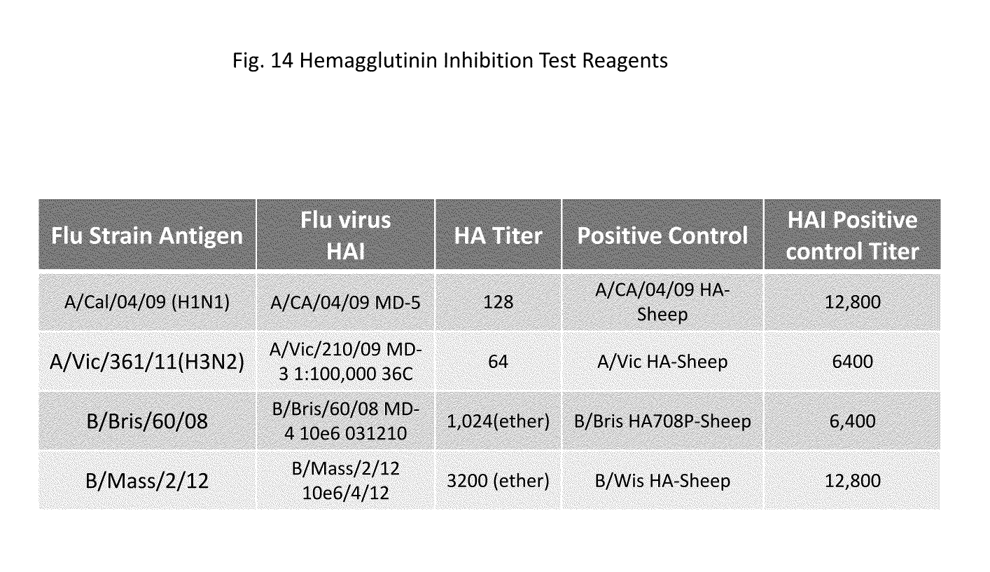

HAI responses to influenza A/California/04/09, A/Victoria/361/11, B/Brisbane/60/08 and B/Massachusetts/2/12 was evaluated on serum samples obtained on day 35. Turkey red blood cells (Lampire Biological Laboratories) were prepared to a 1% suspension in PBS. Serum samples and controls were treated with RDE to inactivate non-specific inhibitors. RDE treated sera were serially diluted in PBS (starting at 1:10) on 96-well V bottom plates. Turkey red blood cells and standardized HA antigens were added to diluted sera and the plate were incubated at room temperature for 45-50 minutes. Inhibition of hemagglutination was determined by tilting the plate to detect the tear-shaped streaming of the red blood cells in the sample wells which flow at the same rate as the red blood cells control wells. The HAI inhibition titers were recorded as the reciprocal of the highest serum dilution where hemagglutination inhibition was observed. The final titer of a serum was reported as the geometric mean (GMT) of the replicate HAI titers. Any sample resulting in a titer less than 10 was assigned a value of 5. (See FIG. 14).

e) Statistical Methods

Results are presented using the geometric mean titer (GMT) and corresponding 95% CI. Pairwise comparisons of vaccine groups were analyzed and estimated by using a two-tailed student's t-test. A p-value<0.05 was considered significant for vaccine group comparisons.

### Example 11

### Results of Mouse Study for Quadrivalent Influenza (Q-Flu) and RSV F Combination Vaccine

Mice were immunized as described in Example 10. RSV F immune responses were assessed by anti-F IgG titer determination using enzyme-linked immunosorbent assay (ELISA), palivizumab competitive ELISA, microneutralization (MN) assay, while influenza immune responses were measured by hemagglutination inhibition assay (HAI), also as described in Example 10.

a) IgG Response

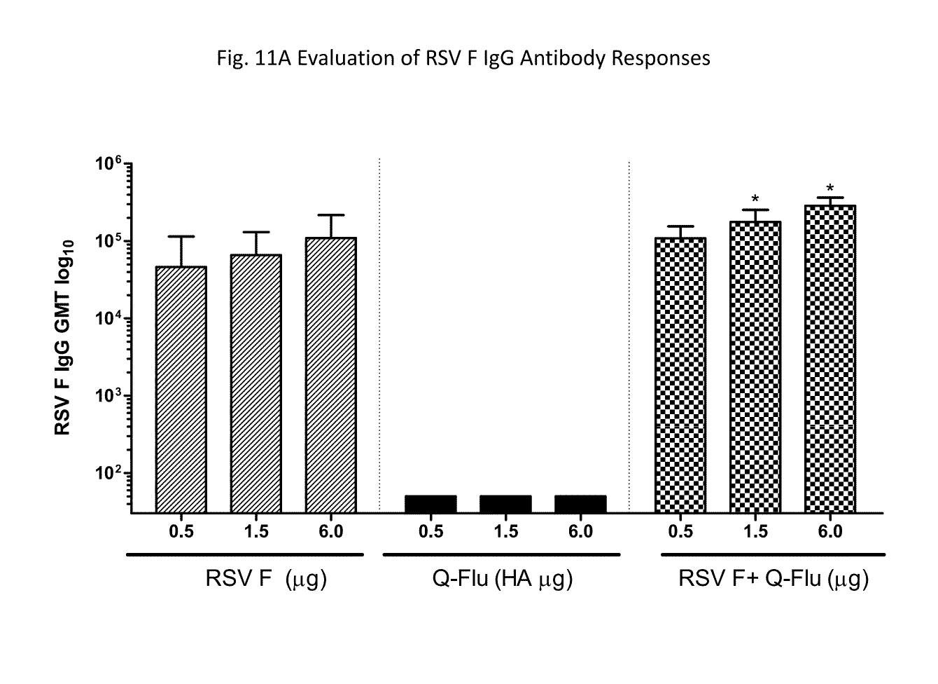

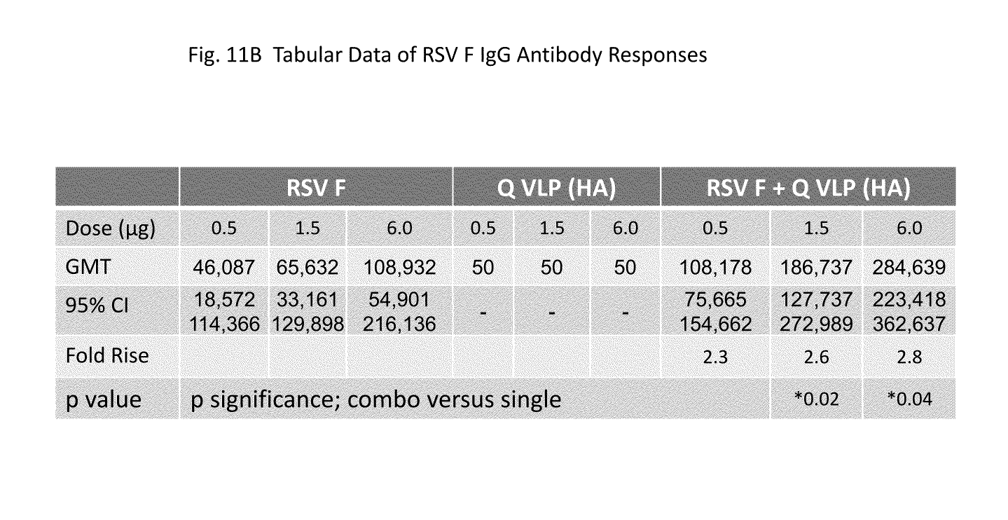

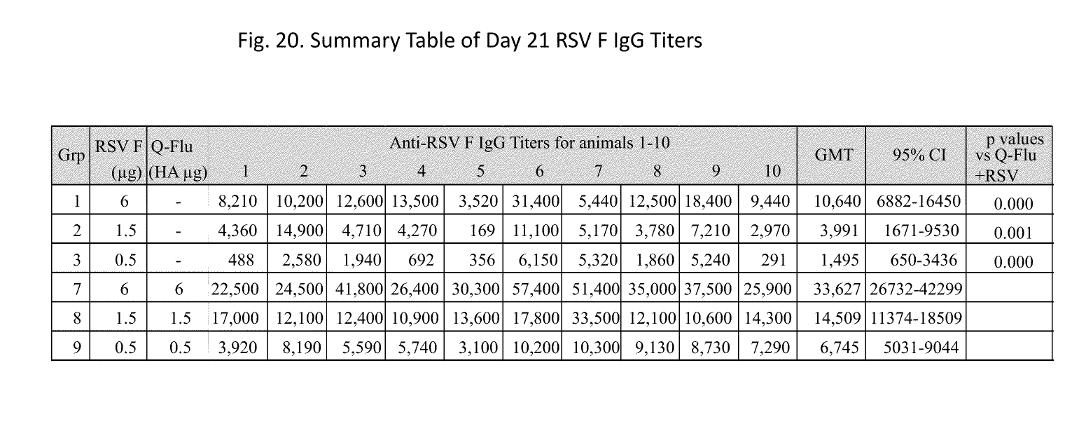

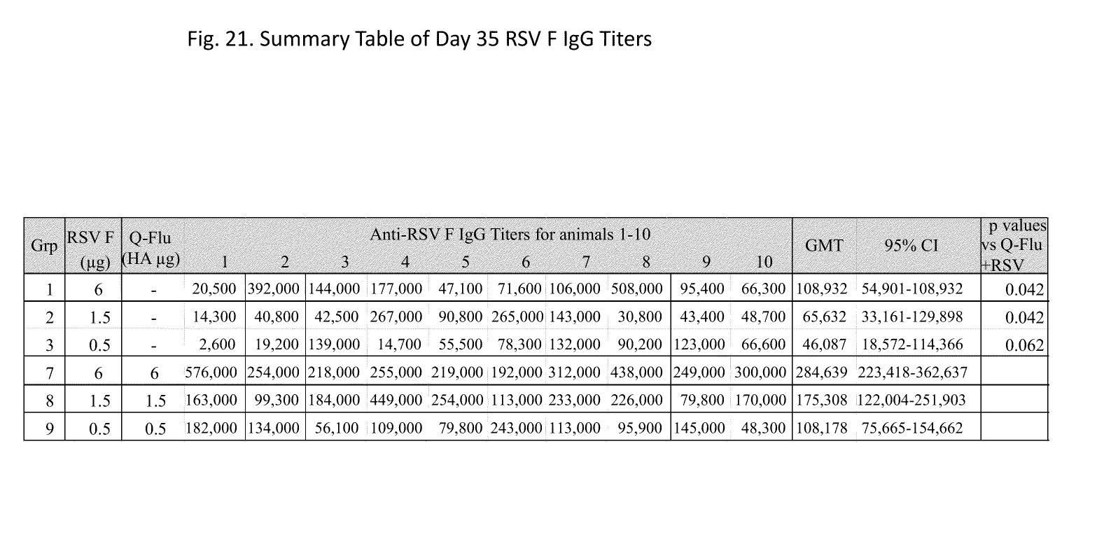

All groups of mice immunized with RSV F single vaccine and combination RSV F/influenza VLP vaccine mounted very high and dose dependent serum IgG responses (FIG. 20). Significant boosting effect was seen with the second immunization for all the groups (p<0.01) (FIGS. 20 and 21). The combination vaccine increased anti-RSV F IgG titers for all the doses compared to RSV F administered alone, with significant difference achieved for 6 μg and 1.5 μg doses with p<0.05 (FIGS. 11A, 11B, and 21). Day 35 anti-RSV F GMT range for RSV F alone was 46,087 to 108,932 whereas the GMT range for the combination vaccine was 108,178 to 284639 (FIG. 21), indicating a 2.3 to 2.8 fold rise. (FIG. 11B).

b) Palivizumab-Competitive Antibodies

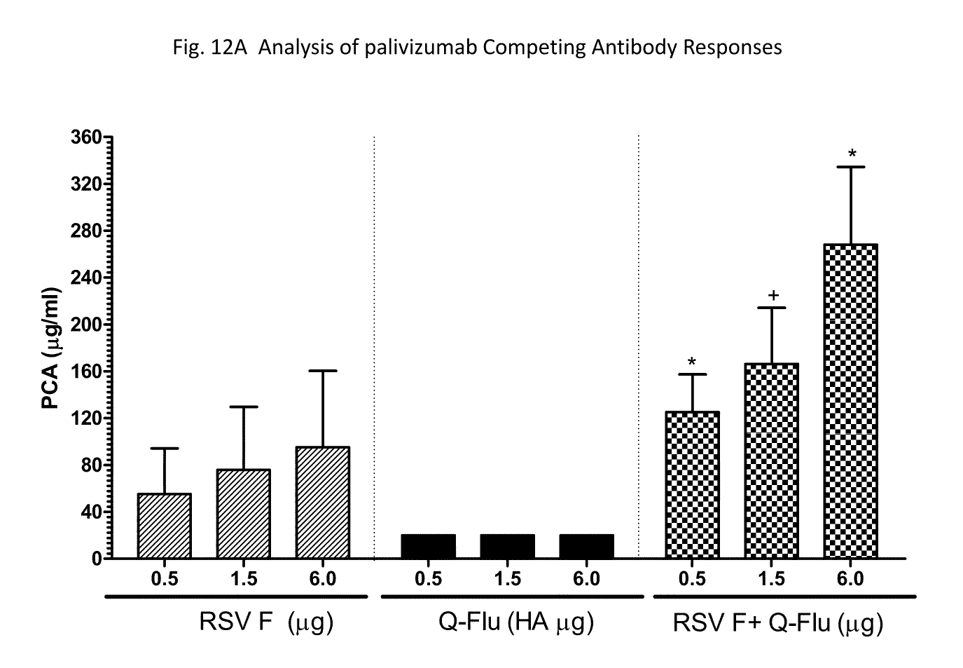

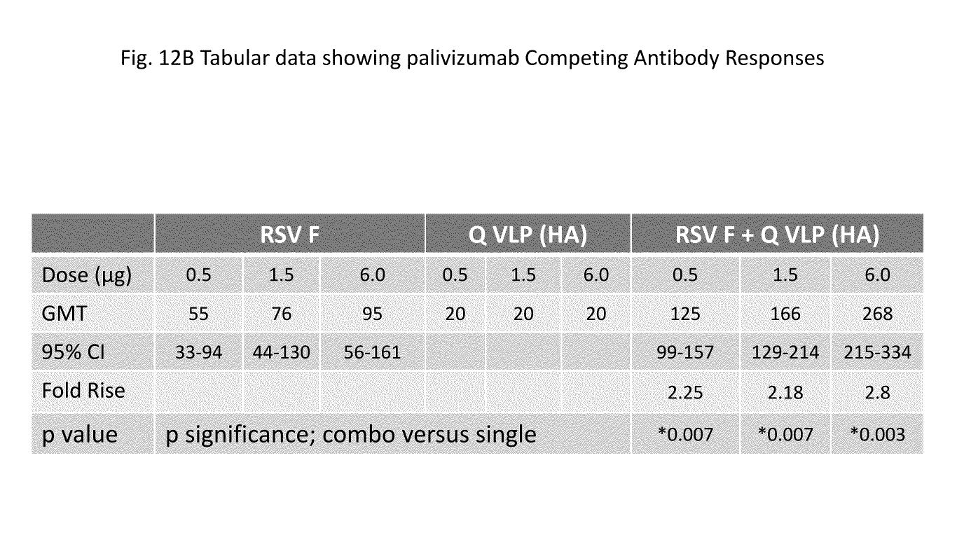

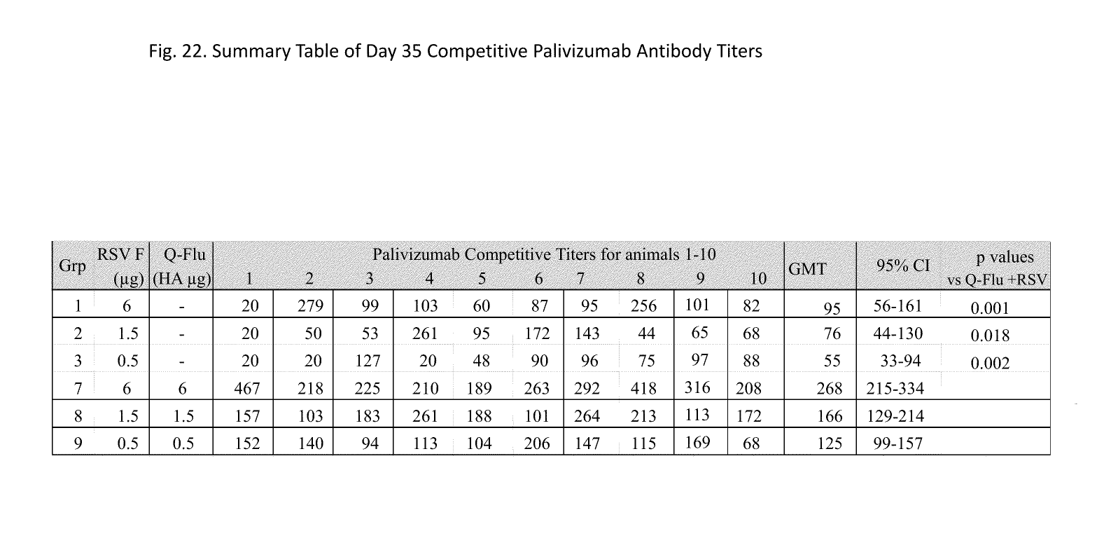

The functional ability of the antibodies generated with all the vaccination groups was determined by competitive ELISA set to indicate the 50% inhibition titers against palivizumab. Similarly to RSVF IgG responses, palivizumab competitive antibody (PCA) titers were also significantly higher (p<0.05) for the group that received combination RSV F/influenza VLP vaccine compared to the groups that received RSV F alone (FIGS. 12A, 12B, and 22). PCA range for RSV F alone was 55 to 95 μg/ml whereas the range for the combination vaccine was 125 to 268 μg/ml (FIG. 22) indicating a 2.2 to 2.8 fold rise. (FIG. 12B).

c) RSV-F Neutralizing Antibodies

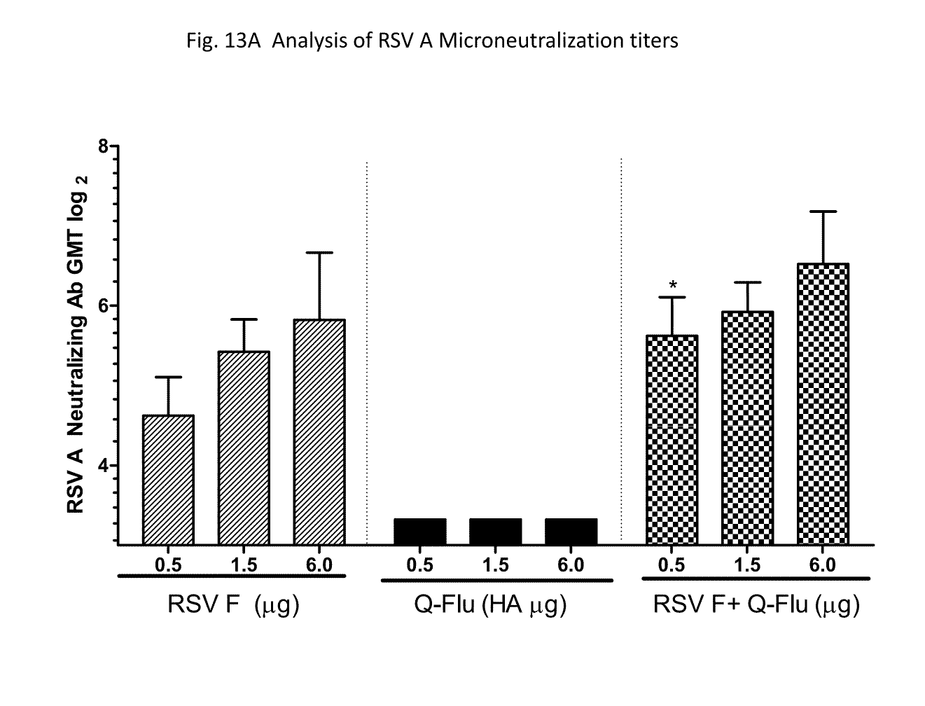

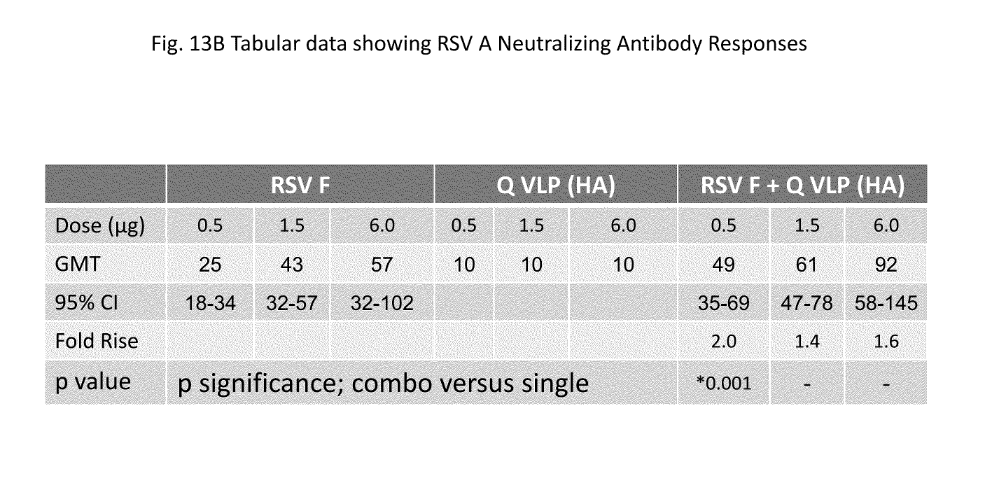

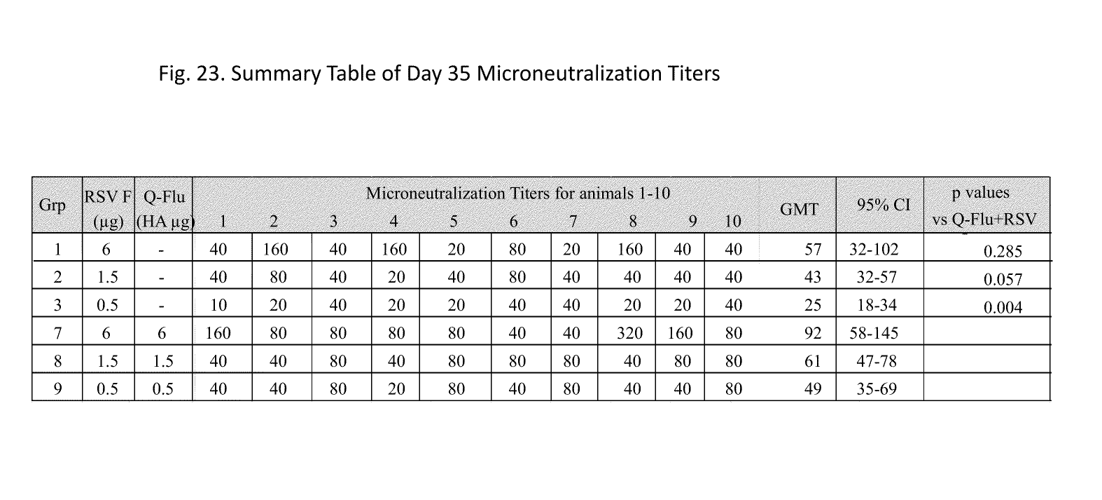

To determine the ability of the antibodies to neutralize RSV virus, microneutralization assay was performed. All groups generated high levels of neutralizing titers in a dose dependent manner (FIGS. 13A and 13B). The combination vaccine increased the titers for all the groups that received the combination vaccine with significant difference achieved for 0.5 μg dose (p<0.01) (FIG. 23).

d) Influenza Specific Responses

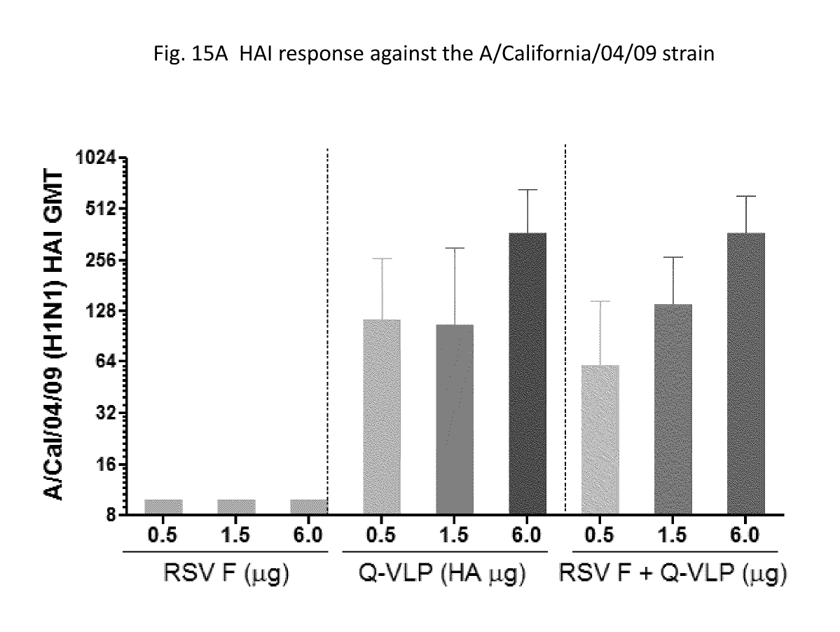

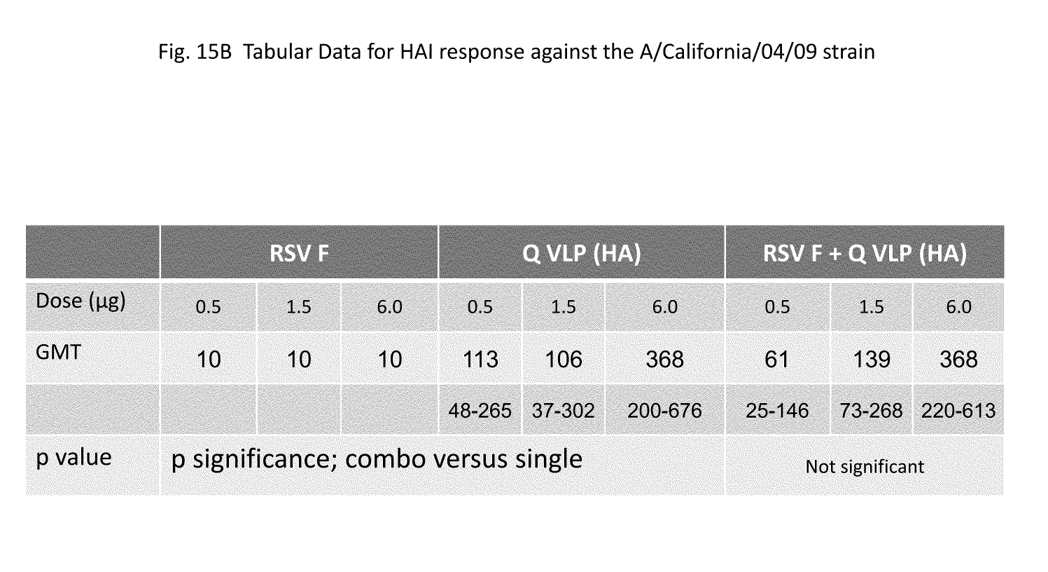

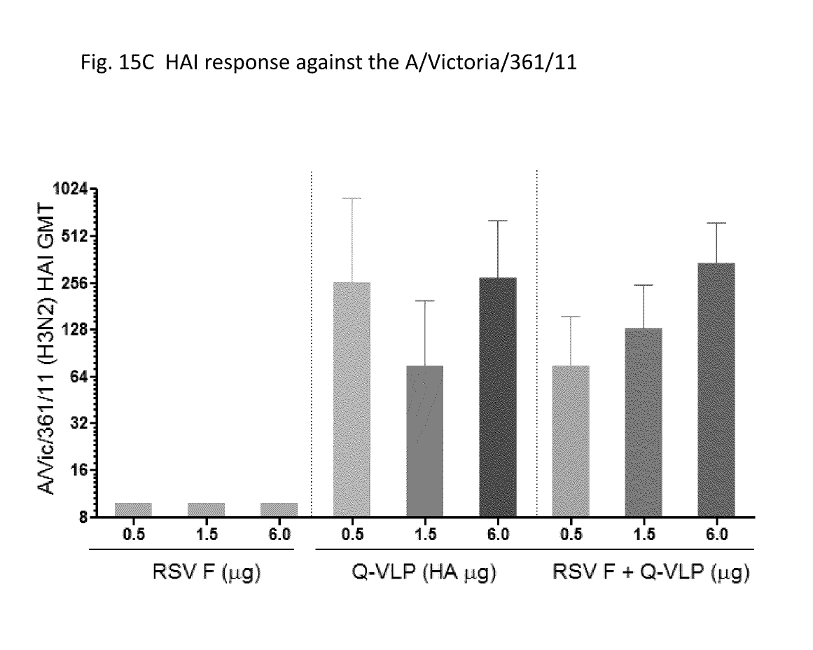

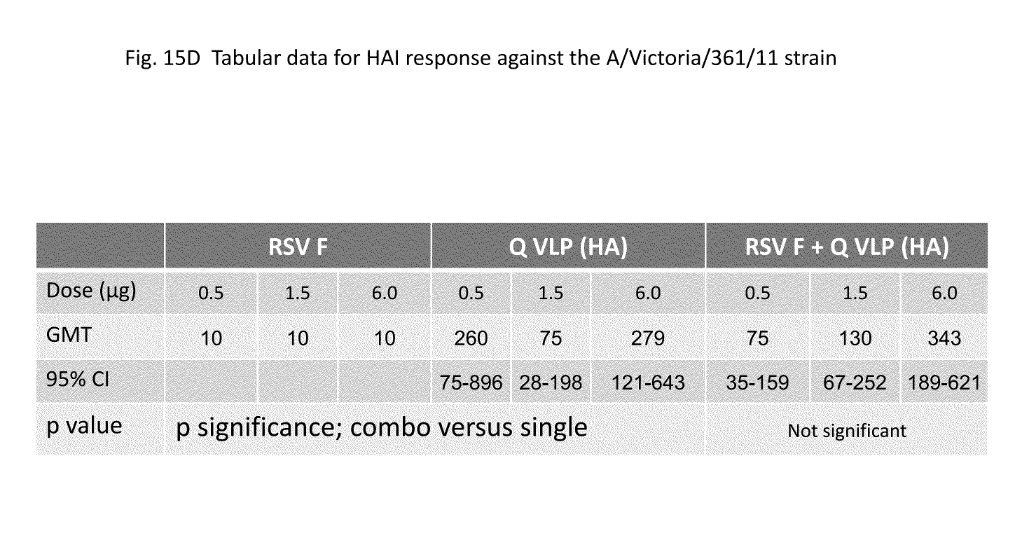

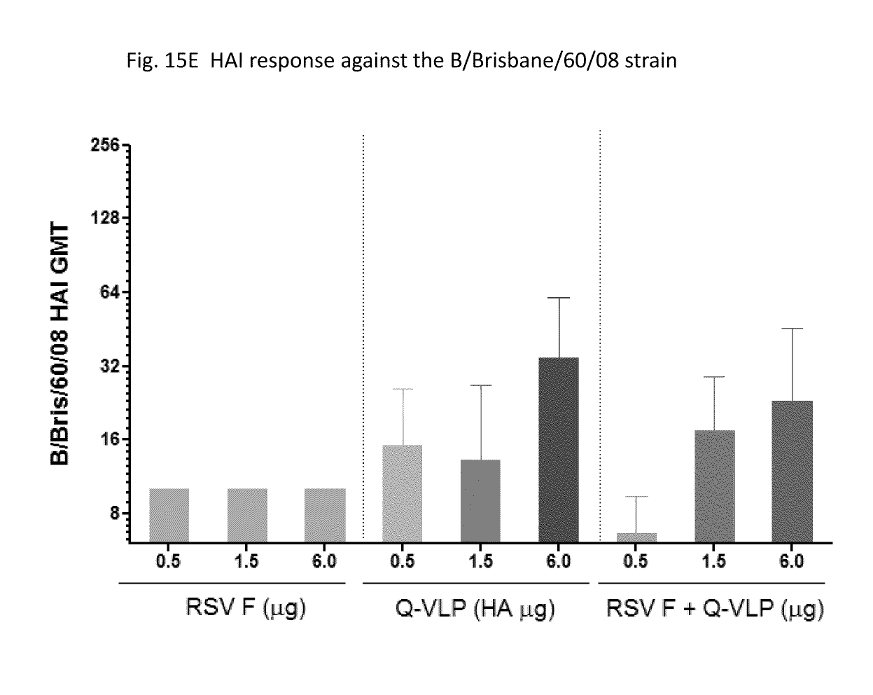

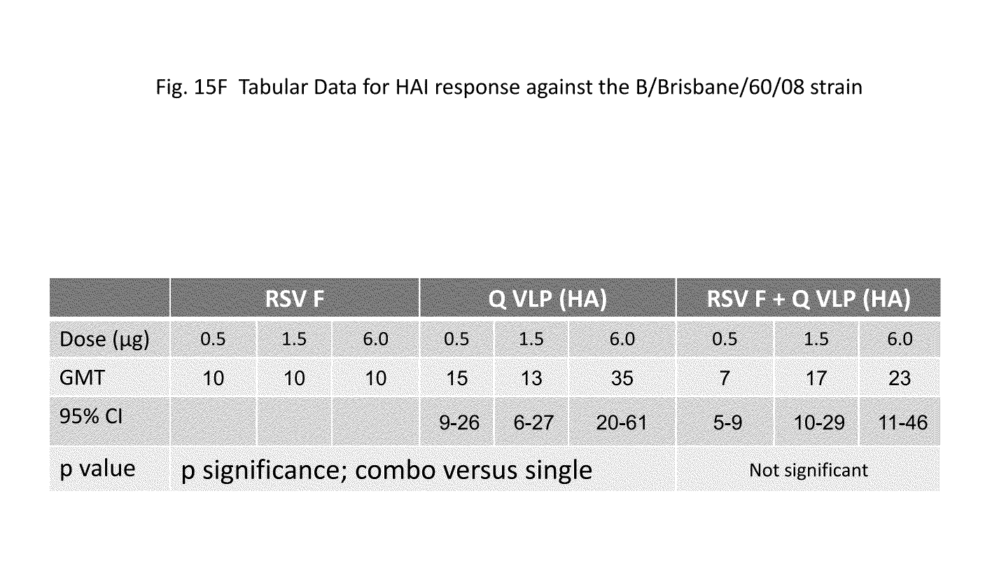

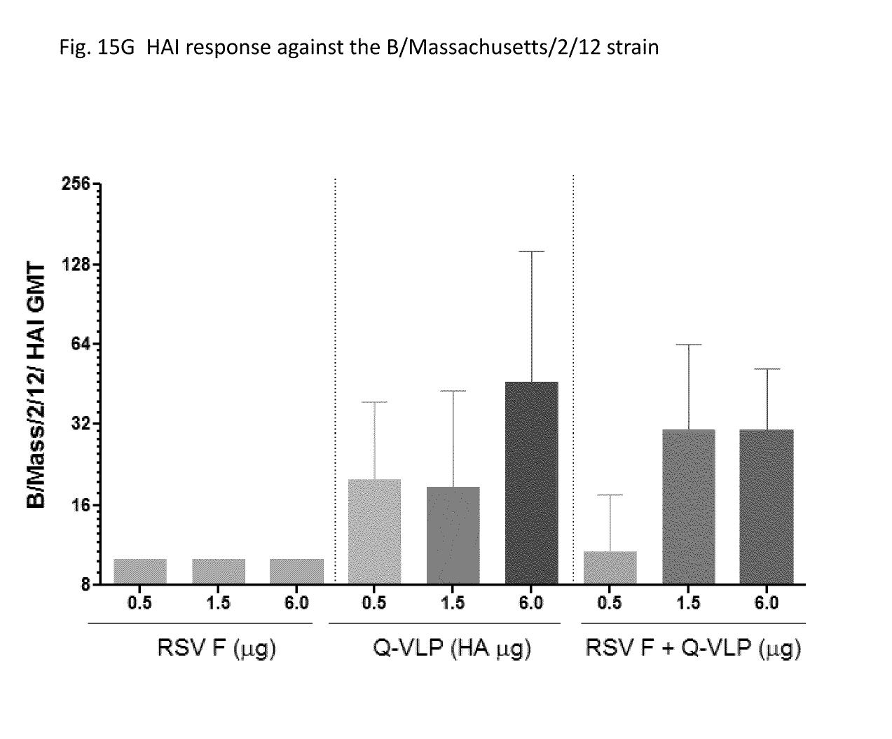

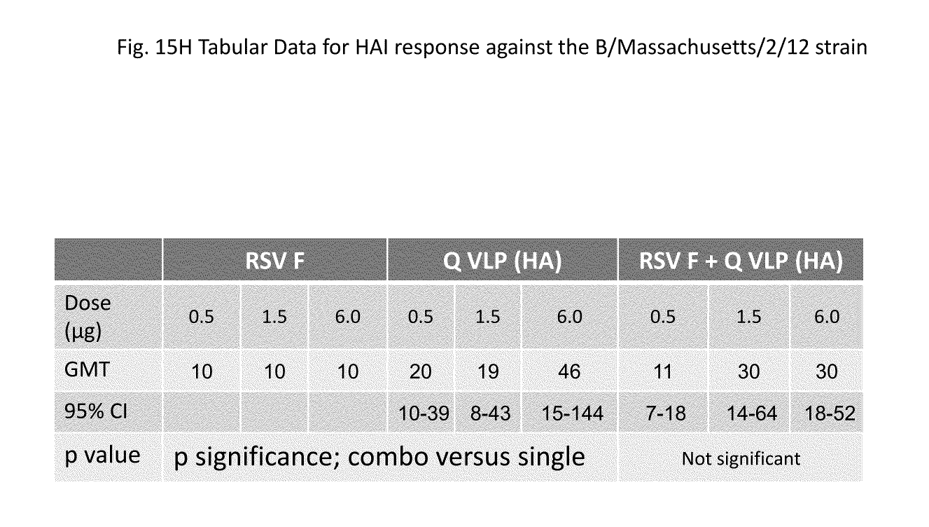

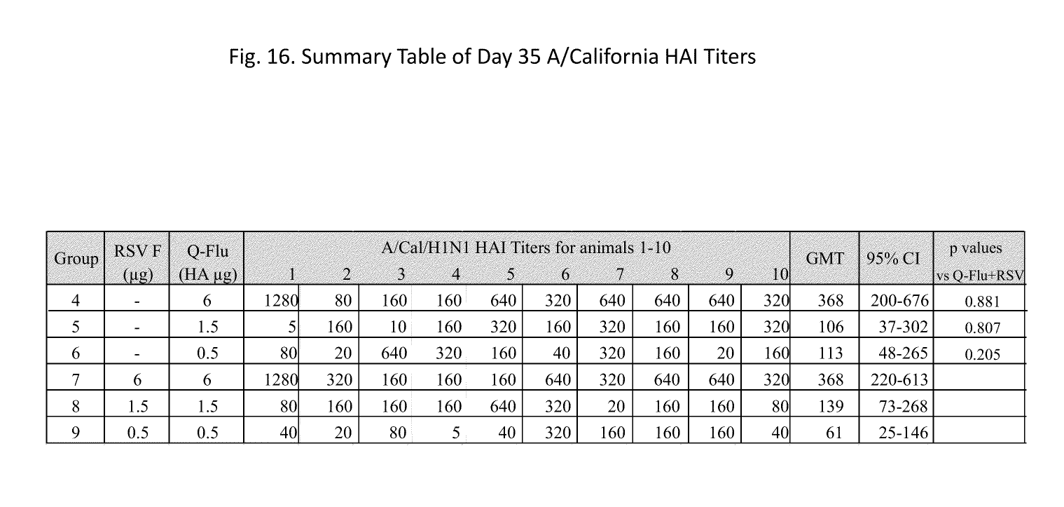

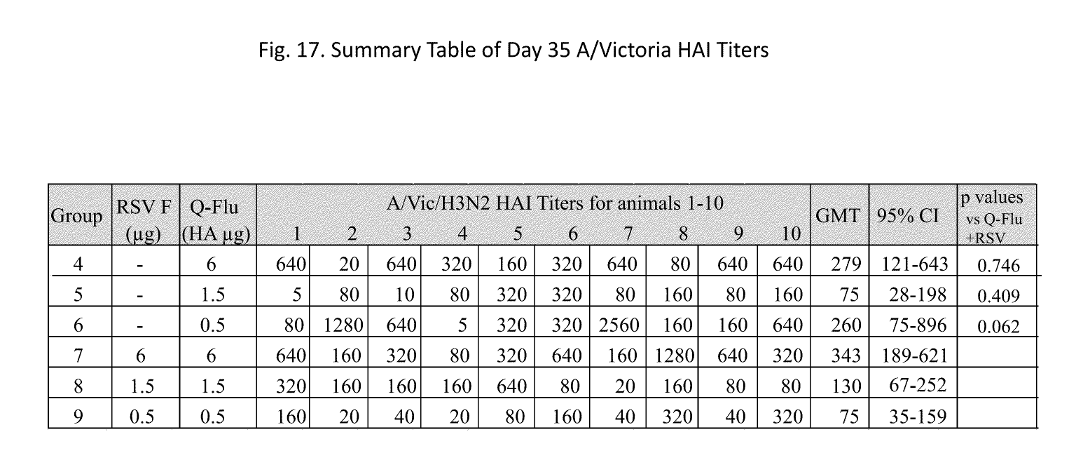

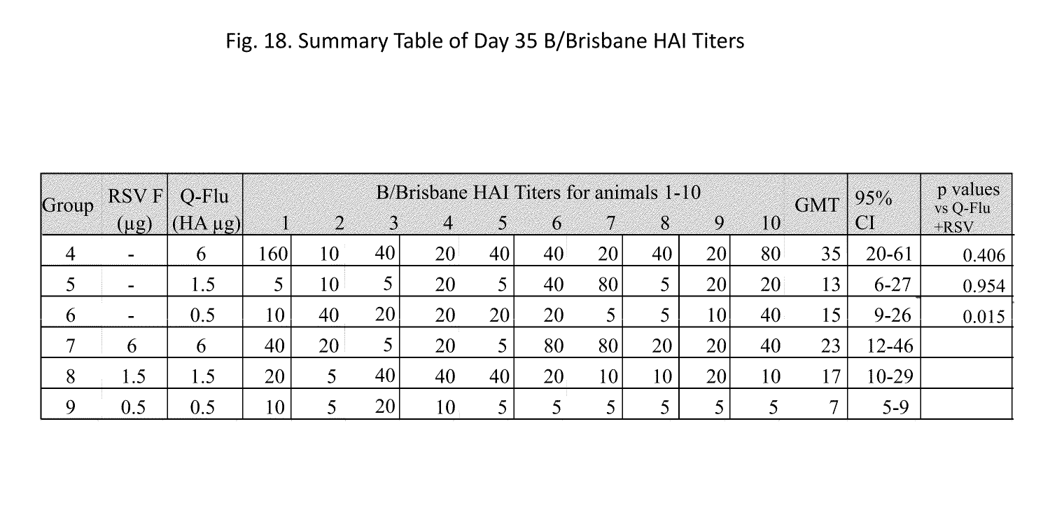

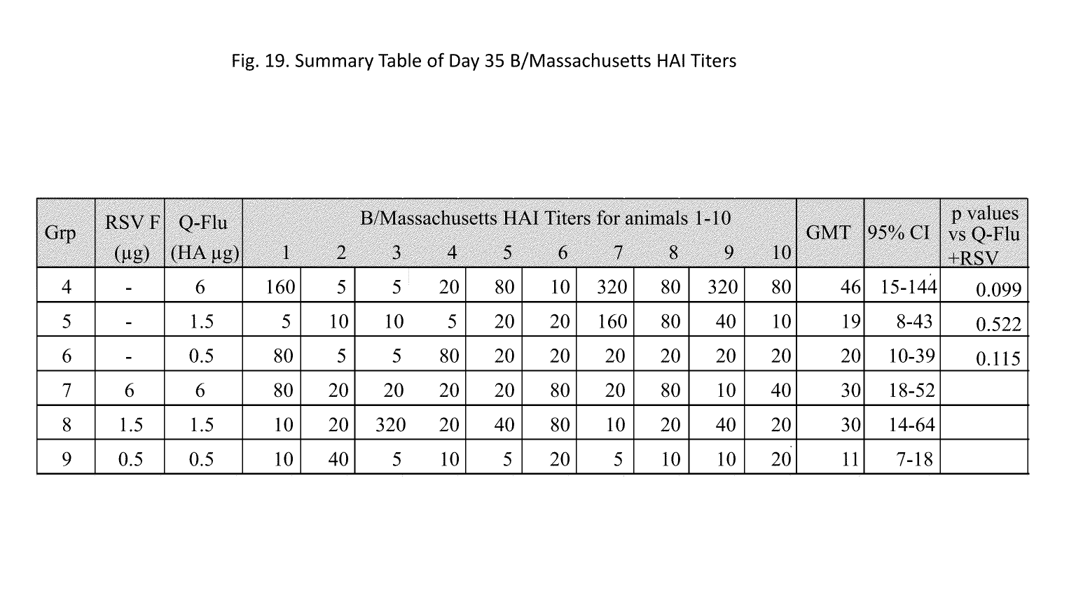

To evaluate influenza specific responses, HAI titers were determined for all four individual strains, A/California/04/09 (H1N1) (See FIGS. 15A, 15B, and 16), A/Victoria/361/11 (H3N2) (See FIGS. 15C, 15D, and 17) and B/Brisbane/60/08 (See FIGS. 15E, 15F, and 18) and B/Massachusetts/2/12 (See FIGS. 15G, 15H, and 19). HAI titers were high despite the fact that the final Q-Flu preparation contained only 1.5 gig, 0.375 gig, or 0.125 μg of each strain. In contrast to RSV F responses, HAI responses with Q-Flu vaccine alone was not significantly different than those with the combination RSV F/influenza VLP vaccine (FIGS. 15-19). This result suggested that the immunogenicity of influenza VLP vaccine was not reduced by the co-administration of RSV F.

e) Fold Rise Comparison

Table 6 below illustrates the potentiating effect of combining both the RSV component and the Q-flu component on the magnitude and character of the anti-RSV-F response. Note that in Study ID No. 27 the A/Perth strain was used whereas in study 46, A/Victoria/361/11 strain was used.

TABLE 6
Ratio Combination Composition RSV response
to Single Composition RSV response

| | RSV Study ID No. 27 | | RSV Study Id No. 46 | | |
|---|---|---|---|---|---|
| | Combo/Single | | Combo/Single | | |
| Antigen Dose (μg) | 3.0 | 9.0 | 0.5 | 1.5 | 6.0 |
| RSV F- IgG | 2.4 | 2.3 | 2.3 | 2.6 | 2.8 |
| RSV A- Neut | 1.1 | 2.0 | 2.0 | 1.4 | 1.6 |
| PCA | 2.5 | 2.6 | 2.3 | 2.2 | 2.8 |

The foregoing detailed description has been given for clearness of understanding only and no unnecessary limitations should be understood therefrom as modifications will be obvious to those skilled in the art.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

1. An immunogenic composition comprising a respiratory syncytial virus (RSV) fusion (F) component and an influenza component.

2. The composition of claim 1 wherein the RSV F component comprises an RSV F protein.

3. The composition of claim 1 wherein the influenza component comprises an influenza Virus-Like Particle (VLP).

4. The composition of claim 3 wherein the influenza VLP comprises an influenza HA protein, an influenza NA protein, and influenza M1 protein.

5. The composition of claim 1 further comprising two, three or four influenza VLPs, wherein each VLP comprises an HA protein from a different strain.

6. The composition of claim 5 wherein each influenza VLP further comprises an NA protein from the same strain as the HA protein.

7. The composition of claim 5 wherein each influenza VLP comprises an M1 protein derived from the influenza strain A/Indonesia/5/05.

8. An immunogenic composition comprising a respiratory syncytial virus (RSV) fusion (F) component and three influenza components,
wherein each influenza component comprises a VLP,
wherein each VLP comprises an influenza M1 protein, an influenza NA protein, and an influenza HA protein; and
wherein the NA protein and the HA protein in each VLP are derived from the same influenza strain,
wherein the influenza proteins in the first, second, and third VLPs are derived from different strains than each other.
wherein the first, second and third VLPs each comprise an M1 protein derived from the same strain.

9. The immunogenic composition of claim 8 further comprising a fourth influenza component, wherein the fourth influenza component comprises a VLP, wherein the fourth VLP comprises an HA protein and an NA protein derived from a different strain than the strain for the first, second and third VLPs; and wherein the M1 protein is derived from the same strain as the first, second and third VLPs.

10. The immunogenic composition of claim 8 wherein the M1 protein is derived from an avian influenza virus.

11. The immunogenic composition of claim 10 wherein the avian influenza virus is the A/Indonesia/5/05 influenza virus strain.

12. The composition of claim 2 wherein the RSV F protein comprises a mutation that inactivates the primary cleavage site or the secondary cleavage site.

13. The composition of claim 2 wherein the RSV F protein comprises a primary cleavage site inactivated by introducing at least one amino acid substitution at positions corresponding to arginine 133, arginine 135, and arginine 136 of the wild-type RSV F protein (SEQ ID NO: 2).

14. The composition of claim 2 wherein the RSV F protein comprises a deletion of the amino acids corresponding to amino acids 137-146 of the wild-type RSV F protein (SEQ ID NO: 2).

15. The composition of claim 2 wherein the RSV F protein comprises SEQ ID NO:8.

16. A kit comprising a respiratory syncytial virus (RSV) fusion (F) component and at least one influenza component, wherein each component is in a separate container.

17. A method of inducing a protective response against RSV and an influenza strain comprising administering the composition of claim 1.

18. The method of claim 17 wherein administering comprises steps
(a) storing the RSV component in a container at 2-8° C.,
(b) storing the influenza component in a container at 2-8° C.,
(c) mixing RSV component with the influenza component to provide a combination composition; and
(d) injecting the composition into an animal intramuscularly,
whereby a protective immune response against infection by influenza and by RSV is obtained.

19. The method of claim 18 wherein the animal is a human.

20. The method of claim 19 wherein the human is an infant.

21. The method of claim 18 wherein the protective response comprises anti-RSV F neutralizing antibodies.

22. The method of claim 18 wherein the protective response comprises hemagglutination inhibition and the hemagglutination inhibition is greater when the RSV component and influenza component are co-administered compared to administration of each component alone.

23. The method of claim 21 wherein the anti-RSV F neutralizing antibody response is greater when the RSV component and influenza component are co-administered compared to administration of each component alone.

24. The method of claim 18 wherein the protective response comprises an anti-palivizumab antibody response.

25. The method of claim 24 wherein anti-palivizumab antibody response is greater when the RSV component and influenza component are co-administered compared to administration of each component alone.
